(19) United States
(12) Patent Application Publication
(10) Pub. No.: US 2008/0036373 A1
(43) Pub. Date: Feb. 14, 2008

(54) Platinum complex and light-emitting device

(75) Inventors: Hisanori Itoh, Kanagawa (JP); Takeshi Iwata, Kanagawa (JP); Yoshimasa Matsushima, Kanagawa (JP)

Correspondence Address: MCDERMOTT WILL & EMERY LLP, 600 13TH STREET, N.W., WASHINGTON, DC, 20005-3096, US

(73) Assignee: TAKASAGO INTERNATIONAL CORPORATION

(21) Appl. No.: 11/882,976

(22) Filed: Aug. 8, 2007

(30) Foreign Application Priority Data
Aug. 10, 2006 (JP) 2006-218273

(51) Int. Cl. H01L 51/54; C07C 211/00; C07D 215/30; C07D 231/00; C07D 231/02; C07D 401/00; C07D 403/00; C07D 471/04; C07F 15/00; H01J 63/04

(52) U.S. Cl. 313/504; 546/275.4; 546/7; 546/88; 548/356.5; 548/365.1; 548/371.4; 556/137; 564/427; 564/428

(57) ABSTRACT

A platinum complex represented by the general formula (1) below, useful as a phosphorescence emission material, a tetradentate ligand useful for synthesizing the platinum complex, and a light-emitting device containing at least one of the platinum complex.

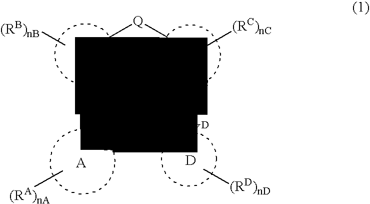

wherein ring B represents a nitrogen-containing aromatic heterocyclic ring; rings A, C and D each independently represent an aromatic or aromatic heterocyclic ring, while either of the rings C and D represents five-membered ring, the other represents a five- or six-membered ring; R^{A}, R^{B}, R^{C}, and R^{D} respectively represent substituents on the rings A, B, C, and D; two of X^{A}, X^{B}, X^{C}, and X^{D} represent a nitrogen atom that may be bound with the platinum atom by a coordinate bond, the others each independently represent a carbon atom or nitrogen atom that may be bound with the platinum atom by a covalent bond; Q represents a bivalent atom or atomic group bridging the rings B and C; and Y^{C} and Y^{D} each independently represent a carbon atom or nitrogen atom.

### BACKGROUND OF THE INVENTION

1. Field of the Invention

The present invention relates to a new platinum complex useful, for example, as a light-emitting material and so on and a light-emitting device using the complex. Further, the present invention, minutely, relates to a new platinum complex usable, for example, as a light-emitting material and so on in the fields such as a display device, a display, a backlight, an electrophotographic machine, an illumination light source, a recording light source, an exposure light source, a reading light source, a sign and mark, a signboard, and interior goods; and a light-emitting device using the complex.

2. Description of the Related Art

Researches and developments on various display devices are intensively carried out recently, and among them, an organic electroluminescent device (hereinafter, referred to as “organic EL device”), which emits high-brightness light at low voltage, is attracting the interests as a promising next-generation display device.

Various materials have been used for a light-emitting material of the organic EL device and use of a phosphorescent material in the light-emitting layer of the organic EL device was proposed as a means for improving the light-emitting characteristics. As the organic EL devices containing a metal complex having such properties as the phosphorescent material, devices using various complexes having iridium as the heavy metal have been so far developed. In addition, there are some scattered reports on devices containing complexes having platinum as the heavy metal recently.

There was reported, for example, that an ortho-metalated platinum complex, in which a compound having an arylpyridine skeleton was used as the ligand and platinum was used as the heavy atom, was useful as a phosphorescence-emitting material. In addition, there was also reported that a platinum complex in which a bipyridine/biaryl skeleton compound was used as the ligand (US 2002/0068190 A1).

On the other hand, a platinum complex having a tetradentate ligand obtained by introducing a phenol group into a bipyridine or phenanthroline skeleton was reported (U.S. Pat. No. 6,653,654) and a platinum complex having a tetradentate ligand which have a bridged phenylpyridine skeleton by a bridging group was reported recently (EP 1683804 A1, US 2006/0182992 A1, US 2006/0073359 A1, US 2007/0082284 A1).

As the emitting light of the organic EL device using the phosphorescence luminescence is limited to red and green under the present situation, the coverage of the organic EL device for the color display using phosphorescence luminescence is narrow. Therefore the development of the device having improved luminescence characteristic for other colors is hoped. Especially, if the luminescence characteristics of the light emission element having a color range of blue to green are improved, it will be able to produce the full-color display and to make the emitting light from organic EL devices white, and greatly advances the practical use of phosphorescence organic EL device.

The compound including iridium (Ir) is actively developed as phosphorescence luminescence complex, and Ir (ppy)₃ is known for a green light emission device under the present situation (Inorganic Chemistry, 30(1991), 1685-1687). On the other hand, FIrpic is known as a phosphorescence luminescence complex that emits blue light, but it is not practicable in the lifetime of the device when using it for the organic EL device and from the point of efficiency (Applied Physics Letters, vol. 79, No. 13 (2001), 2082).

As described above, the research is fairly under way, and there are still many problems to be solved. Especially, concerning the emission wavelength, for example, the development of phosphorescence luminescence material which is required to emit light having a color range from green to blue in the short wavelength area is assumed extremely difficult. To solve these problems, there exists a need for development of a new phosphorescence-emitting material in the short wavelength and further an efficient supplying method of the materials.

### SUMMARY OF THE INVENTION

An object of the present invention, which was made in view of the problems above, is to provide a platinum complex having short wavelength emitting property and useful, for example, as a material for light-emitting devices, and a light-emitting device using the complex.

After intensive studies to overcome the problems above, the present inventors have found that a platinum complex represented by the following General Formula (1) (hereinafter, referred to as “platinum complex of the present invention”) was superior in short wavelength emitting property. After further studies for preparation of devices based on the finding, they also found that the platinum complex was quite favorable as a phosphorescence-emitting material for light-emitting devices, and completed the present invention.

Accordingly, the present invention relates to a platinum complex represented by General Formula (1):

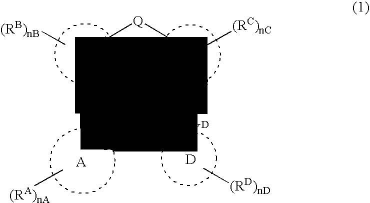

wherein ring A represents a six-membered aromatic ring or a six-membered aromatic heterocyclic ring; ring B represents a five-membered nitrogen-containing aromatic heterocyclic ring; rings C and D each independently represent an aromatic ring or an aromatic heterocyclic ring, while either of the ring C or D represents five-membered ring, the other represents a five- or six-membered ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C} or R^{D} to form a fused ring independently; two of X^{A}, X^{B}, X^{C}, and X^{D} represent a Nitrogen Atom that may be bound with the platinum atom by a coordinate bond, while the other two each independently represent a carbon atom or nitrogen atom that may be bound with the platinum atom by a covalent bond; Q represents a bivalent atom or atomic group bridging the rings B and C; the ring B and Q, and the ring C and Q each independently may be bound each other via a substituent R^{B} or R^{C} to form a fused ring; Y^{c} and Y^{D} each independently represent a carbon atom or a nitrogen atom; nA is an integer of 0 to 4, nB is an integer of 0 to 2, nC is an integer of 0 to 2 when the ring C is five-membered ring or an integer of 0 to 3 when the ring C is six-membered ring, and nD is an integer of to 3 when the ring D is five-membered ring or an integer of 0 to 4 when the ring D is six-membered ring; and when nA, nB, nC or nD is 2 or more, the groups R^{A}, the groups R^{B}, the groups R^{C}, and the groups R^{D} each independently may be bound each other to form a fused ring.

Further, the present invention relates to a light-emitting device containing one or more of the platinum complexes represented by General Formula (1) above.

Furthermore, the present invention relates to a compound represented by General Formula (2):

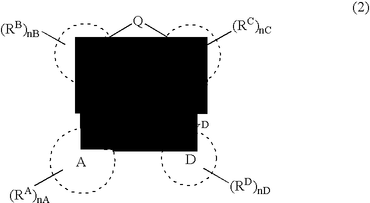

wherein ring A represents a six-membered aromatic ring or a six-membered aromatic heterocyclic ring; ring B represents a five-membered nitrogen-containing aromatic heterocyclic ring; rings C and D each independently represent an aromatic ring or an aromatic heterocyclic ring, while either of the ring C or D represents five-membered ring, the other represents a five- or six-membered ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C}, or R^{D} to form a fused ring independently; two of X^{A′}, X^{B′}, X^{C′}, and X^{D′} represent a nitrogen atom having a lone electron pair, while the other two represent CH or NH; Q represents a bivalent atom or atomic group bridging the rings B and C; and the ring B and Q, and the ring C and Q each independently may be bound each other via a substituent R^{B} or R^{C} to form a fused ring; Y^{C} and Y^{D} represent a carbon atom or a nitrogen atom; nA is an integer of 0 to 4, nB is an integer of 0 to 2, nC is an integer of 0 to 2 when the ring C is five-membered ring or an integer of 0 to 3 when the ring C is six-membered ring, and nD is an integer of 0 to 3 when the ring D is five-membered ring or an integer of 0 to 4 when the ring D is six-membered ring; and when nA, nB, nC or nD is 2 or more, the group R^{A}, the group R^{B}, the group R^{C}, and the group R^{D} each independently may be bound each other to form a fused ring.

The platinum complex represented by General Formula (1) of the present invention is superior in shorter wavelength compared with a platinum complex as the existing phosphorescence-emitting material, and useful as a phosphorescence-emitting material being able to be used favorably in various light-emitting devices including organic EL devices. Further, the compound represented by General Formula (2) is useful as a tetradentate ligand for use in synthesis of metal complexes including the platinum complexes represented by General Formula (1).

Moreover, there is no report example in platinum complex that has the nitrogen-carbon bond in the uniting part of ring B and ring A yet. In addition, it was found that the luminescence wavelength had been made shorten compared with reported complexes so far by the existence of this nitrogen-carbon bond in platinum complex of the present invention. Thus, the platinum complex of the present invention is a new compound, and may be adjusted the short wavelength or more luminescence compared with existing platinum complex having tetradentate ligand.

### BRIEF DESCRIPTION OF THE DRAWING

FIG. 1 is a view illustrating the configuration of the organic EL device used in Examples.

### DESCRIPTION OF THE PREFERRED EMBODIMENTS

Hereinafter, the platinum complex represented by General Formula (1) and the compound represented by General Formula (2) of the present invention will be described in more detail.

As shown in General Formula (1) above, the platinum complex of the present invention is a platinum complex having a tetradentate ligand containing rings A, B, C, and D wherein the ring B and the ring C are bridged by Q.

In addition, the compound represented by General Formula (2) of the present invention is a compound wherein the ring B and the ring C are bridged by Q. The compound represented by General Formula (2) is a favorable compound as a tetradentate ligand used in the synthesis of metal complexes including a platinum complex.

Hereinafter, both the compounds represented by General Formulae (1) and (2) will be referred to simply as the “compounds of the present invention”.

In the compounds of the present invention, two of the rings A, C, and D each independently represents an aromatic ring or an aromatic heterocyclic ring that may have a substituent R^{A}, R^{C}, or R^{D}; and the rest of the rings represents a nitrogen-containing heterocyclic ring that may have a substituent R^{B}, R^{C}, or R^{D}.

Each of the rings A and B, rings B and C, and rings C and D may be bound each other independently via a substituent group R^{A}, R^{B}, R^{C} or R^{D} to form a fused ring. In addition, when Q is a bivalent atomic group, the ring B and Q, and the ring C and Q may be bound each other independently via a substituent R^{B} or R^{C} to form a fused ring.

The aromatic ring or the aromatic heterocyclic ring constituting the rings A, B, C and D in the compounds of the present invention is not particularly limited as long as it is an aromatic ring or an aromatic heterocyclic ring. In addition, the ring B is always a five-membered ring, and the ring A is always a six-membered ring. Preferred examples of the aromatic ring and aromatic heterocyclic ring of the rings A, B, C and D include benzene, furan, thiophene, selenophene, tellurophene, pyrrole, pyridine, pyridazine, pyrimidine, pyrazine, 1,2,3-triazine, 1,2,4-triazine, 1,2,3,4-tetrazine, oxazole, isoxazole, thiazole, isothiazole, pyrazole, imidazole, 1,2,3-oxadiazole, 1,2,5-oxadiazole, 1,2,3-thiadiazole, 1,2,5-thiadiazole, triazole and tetrazole rings shown below, and the like.

These rings may additionally form a fused ring with a ring selected from the group consisting of rings described above. Examples of the fused rings include the benzologues of the respective rings; and typical examples thereof include naphthalene, anthracene, phenanthrene, chrysene, pyrene, benzofuran, isobenzofuran, thianaphthene, isothianaphthene, benzoselenophene, isobenzoselenophene, benzotellurophene, isobenzotellurophene, indole, isoindole, indolidine, quinoline, isoquinoline, cinnoline, phthalazine, quinazoline, quinoxaline, benzotriazine, benzotetrazine, benzoxazole, benzisoxazole, benzothiazole, benzisothiazole, indazole, benzimidazole, benzoxadiazole, benzothiadiazole and benzotriazole rings, and the like.

When the ring B and C is a nitrogen containing five-membered heterocyclic ring in the compounds represented by General Formulae (1) and (2), typical examples of the preferable rings include 1H-pyrrole, indole, isoindole, pyrazole, 1H-indazole, 2H-indazole, imidazole, benzimidazole, triazole and tetrazole rings, and the like. Examples of more preferable rings include 1H-pyrrole, pyrazole and 1H-indazole. In the present invention, as described above, five-membered rings include the fused rings of five-membered rings with another ring as well as five-membered rings.

When each of the rings A, C and D is a six-membered aromatic or aromatic heterocyclic ring independently in the compounds represented by General Formulae (1) and (2), favorable examples of the rings include benzene, pyridine, pyridazine, pyrimidine and 1, 2, 3-triazine rings, and the like. A fused ring formed from a benzene ring and a suitable ring selected from the group consisting of the aromatic rings and aromatic heterocyclic rings described above is also preferable, and typical examples of such rings include naphthalene, anthracene, phenanthrene, chrysene, pyrene, benzofuran, isobenzofuran, thianaphthene, isothianaphthene, benzoselenophene, isobenzoselenophene, benzotellurophene, isobenzotellurophene, indole, isoindole, indolidine, quinoline, isoquinoline, cinnoline, phthalazine, quinazoline, quinoxaline, benzotriazine, benzotetrazine, benzoxazole, benzisoxazole, benzothiazole, benzisothiazole, indazole, benzimidazole, benzoxadiazole, benzothiadiazole and benzotriazole rings, and the like. Examples of still more preferable rings include benzene, naphthalene, benzofuran, isobenzofuran, thianaphthene and isothianaphthene rings, and the like. In the present invention, as described above, six-membered rings include the fused rings of six-membered rings with another ring as well as six-membered rings.

The nitrogen-containing heterocyclic ring constituting the rings A, C and D in the compounds of the present invention is not particularly limited, and preferable examples of the nitrogen-containing heterocyclic ring include the pyridine, pyridazine, pyrimidine, pyrazine, triazine, tetrazine, 2H-pyrrole, 3H-pyrrole, oxazole, isoxazole, thiazole, isothiazole, pyrazole, imidazole, oxadiazole, thiadiazole, triazole, oxatriazole, thiatriazole, tetrazole, 2H-3,4-dihydropyrrole, oxazoline, isooxazoline, thiazoline, isothiazoline, pyrazoline and imidazoline rings, and the like.

The rings above may be bound with a suitable ring selected from the group consisting of the aromatic rings and aromatic heterocyclic rings described above to form a fused ring. Examples of the fused ring include the benzologues of the respective rings, and typical examples thereof include quinoline, isoquinoline, cinnoline, phthalazine, quinazoline, quinoxaline, benzotriazine, benzotetrazine, 1H-isoindole, 3H-indole, benzoxazole, benzisoxazole, benzothiazole, benzisothiazole, indazole, benzimidazole, benzoxadiazole, benzothiadiazole, and benzotriazole rings, and the like.

More preferable examples of the nitrogen-containing heterocyclic ring include pyridine, quinoline, isoquinoline, 2H-pyrrole, 1H-isoindole, 3H-pyrrole, 3H-indole, oxazole, benzoxazole, isoxazole, benzisoxazole, thiazole, benzothiazole, isothiazole, benzisothiazole, pyrazole, indazole, imidazole, benzimidazole, 2H-3,4-dihydropyrrole, oxazoline, isoxazoline, thiazoline, isothiazoline, pyrazoline and imidazoline rings, and the like.

When the rings A, C and D in the compounds represented by General Formulae (1) and (2) each independently represent a nitrogen-containing six-membered heterocyclic ring, each of these rings is preferably a six-membered ring or the benzologue thereof, and examples thereof include the pyridine, isoquinoline, pyrimidine, quinazoline, pyrazine, 1,2,4-triazine, 1,3,5-triazine, and 1,2,3,5-tetrazine rings, and the like; and more preferable rings thereof include pyridine and isoquinoline rings, and the like.

The Q in the compounds of the present invention represents a bivalent atom or atomic group bridging the rings B and C, and the bridging group Q will be described below in detail. As the bivalent atom or atomic group described above, any bivalent atom or atomic group can be used so long as it can bridge the corresponding rings, and examples of the bivalent atom or atomic group include oxy, thio, seleno, telluro, sulfinyl, sulfonyl, imino, phosphinidene, phosphinylidene, methylene, alkenylidene, carbonimidoyl, carbonyl, thiocarbonyl, silylene and borylene groups shown below.

In the above examples, the imino, phosphinidene, phosphinylidene, methylene, alkenylidene, carbonimidoyl, silylene and borylene groups may be substituted with a suitable substituent. Examples of the substituted imino groups include imino groups in which the hydrogen atom on the nitrogen atom is substituted with a substituent such as imino-protecting group. The imino protecting group may be any one of the protecting groups described, for example, in the literature of “PROTECTIVE GROUPS IN ORGANIC SYNTHESIS Third Edition”, JOHN WILEY & SONS, INC. Typical examples of substituents and protecting groups of the imino group include alkyl, aryl, aralkyl, acyl, alkoxycarbonyl, aryloxycarbonyl, aralkyloxycarbonyl and sulfonyl groups, and the like.

Typical examples of the alkyl group-substituted imino groups, i.e., alkylimino groups, include N-methylimino, N-ethylimino, N-isopropylimino and N-cyclohexylimino groups, and the like.

Typical examples of the aryl group-substituted imino group, i.e., arylimino groups, include N-phenylimino, N-(2,4,6-trimethylphenyl)imino, N-(2,6-diisopropylphenyl)imino, N-(3,5-di-tert-butylphenyl)imino, N-(1-naphthyl)imino, N-(2-naphthyl)imino, N-(9H-carbazole-9-yl)imino and N-(9-anthryl)imino groups, and the like.

Typical examples of the aralkyl group-substituted imino groups, i.e., aralkylimino groups, include N-benzylimino and N-(1-phenylethyl)imino groups, and the like.

Typical examples of the acyl group-substituted imino groups, i.e., acylimino groups, include formylimino, acetylimino, propionylimino, acryloylimino, pivaloylimino, pentanoylimino, hexanoylimino and benzoylimino groups, and the like.

Typical examples of the alkoxycarbonyl group-substituted imino groups, i.e., alkoxycarbonylimino groups, include methoxycarbonylimino, ethoxycarbonylimino, n-propoxycarbonylimino, n-butoxycarbonylimino, tert-butoxycarbonylimino, pentyloxycarbonylimino and hexyloxycarbonylimino groups, and the like.

Typical examples of the aryloxycarbonyl group-substituted imino groups, i.e., aryloxycarbonylimino groups, include phenoxycarbonylimino and 2-naphthyloxycarbonylimino groups, and the like.

Typical examples of the aralkyloxycarbonyl group-substituted imino groups, i.e., aralkyloxycarbonylimino groups, include a benzyloxycarbonylimino group and the like.

Typical examples of the sulfonyl group-substituted imino groups, i.e., sulfonylimino groups, include a methanesulfonylimino and p-toluenesulfonylimino groups, and the like.

The phosphinidene group that may have a substituent group is, for example, a phosphinidene group in which the hydrogen atom on the phosphorus atom is substituted with a substituent such as a hydrocarbyl group; and typical examples thereof include methylphosphinidene, ethylphosphinidene, isopropylphosphinidene, phenylphosphinidene and benzylphosphinidene groups, and the like.

The phosphinylidene group that may have a substituent group is, for example, a phosphinylidene group in which the hydrogen atom on the phosphorus atom is substituted with a substituent such as a hydrocarbyl group; and typical examples thereof include methylphosphinylidene, ethylphosphinylidene, isopropylphosphinylidene, phenylphosphinylidene and benzylphosphinylidene groups, and the like.

The methylene group that may have a substituent group is, for example, a methylene group in which at least one hydrogen atom on the carbon atom is substituted with a substituent such as a hydrocarbyl group, alkoxy group, acyloxy group, alkylthio group, cyano group and a halogen atom; and typical examples thereof include ethane-1,1-diyl, propane-1,1-diyl, propane-2,2-diyl, benzylidene, 1-phenylethane-1,1-diyl, diphenylmethylene, 1,3-diphenylpropane-2,2-diyl, dimethoxymethylene, diethoxymethylene, diacetoxymethylene, di(methylthio)methylene, di(ethylthio)methylene, dicyanomethylene and difluoromethylene groups, and the like.

The alkenylidene group that may have a substituent is, for example, an alkenylidene group in which at least one hydrogen atom on the carbon atom is substituted with a substituent group such as a hydrocarbyl group, a cyano group or a halogen atom; and typical examples thereof include propen-1,1-diyl, 2-methylpropen-1,1-diyl, 2-phenylethen-1,1-diyl, 2,2-diphenylethen-1,1-diyl, 3-phenyl-1-propen-1,1-diyl, 2,2-dicyanoethen-1,1-diyl and 2,2-difluoroethen-1,1-diyl groups, and the like.

The carbonimidoyl group that may have a substituent is, for example, a carbonimidoyl group in which the hydrogen atom on the nitrogen atom is substituted with a substituent such as the hydrocarbyl group; and typical examples thereof include N-methylcarbonimidoyl, N-phenylcarbonimidoyl and N-benzylcarbonimidoyl groups, and the like.

The silylene group that may have a substituent is, for example, a silylene group in which at least one hydrogen atom on the silicon atom is substituted with a substituent such as a hydrocarbyl group; and typical examples thereof include dimethylsilylene, diethylsilylene, methylphenylsilylene, diphenylsilylene, dibenzylsilylene, silole-1,1-diyl and dibenzosilole-9,9-diyl group, and the like.

Examples of the borylene groups that may have a substituent include a (2,4,6-trimethylphenyl)borylene group and the like.

In addition, when the bivalent atomic group has two or more substituents, they may bind each other to form a ring independently. Typical examples of the rings formed include cyclopropan-1,1-diyl, cyclobutan-1,1-diyl, cyclopentan-1,1-diyl, cyclohexan-1,1-diyl, 9H-fluoren-9,9-diyl, 1,3-dioxolan-2,2-diyl, 1,3-dioxan-2,2-diyl, 1,3-dithiolan-2,2-diyl, 1,3-dithian-2,2-diyl and 9H-silafluoren-9,9-diyl groups, and the like. The formed ring may be substituted additionally with a suitable substituent, for example, a substituent described in the substituents R^{A} to R^{D} below.

In addition, preferable examples of bivalent atoms or atomic groups constituting Q also include a bivalent atomic group formed by binding in series or condensing of two to five of the bivalent atoms and atomic groups selected from the groups above. Example forms of series bonds presented by names and structural formulae include as follows; ethylene group: —CH₂CH₂—, cis-ethene-1,2-diyl group: —CH═CH—, trimethylene group: —CH₂CH₂CH₂—, phenylene group: —C₆H₄—, ethylenedioxy group: —OCH₂CH₂O—, trimethylenedioxy group: —OCH₂CH₂CH₂O—, phenylenedioxy group: —OC₆H₄O—, carbonyloxy group: —O(C═O)—, carbonyldioxy group: —O(C═O)O—, carbonylthio group: —S(C═O)—, carbonyldithio group: —S(C═O)S—, carbonylimino group: —NR(C═O)—, carbonyldiimino group: —NR(C═O)R—, thiocarbonyloxy group: —O(C═S)—, thiocarbonyldioxy group: —O(C═S)O—, thiocarbonylthio group: —S(C═S)—, thiocarbonyldithio group: —S(C═S)S—, thiocarbonylimino group: —NR(C═S)—, thiocarbonyldiimino group: —NR(C═S)NR—, silylenedioxy group: —O(SiR₂)O—, and the like. The bivalent atomic group formed by binding in series or condensation may be substituted by suitable substituent or substituents and when plural substituents exist on the atoms and/or atomic groups it may be independently bound each other to form a ring.

Examples of more preferable bivalent atom or atomic groups constituting the group Q include oxy group, thio group, sulfonyl group, imino group that may have a substituent, methylene group that may have a substituent, alkenylidene group that may have a substituent, carbonyl group, thiocarbonyl group, silylene group that may have a substituent, and the like.

Y^{C} and Y^{D} in the compound represented by the general formulae (1) and (2) of the present invention represent a carbon atom or a nitrogen atom adjoining the atoms X^{C} and X^{D} constituting the ring C or ring D in the same ring. However Y^{C} and Y^{D} never represent simultaneously a nitrogen atom.

The groups R^{A}, R^{B}, R^{C}, and R^{D} in the compound of the present invention represent substituents respectively on rings A, B, C and D. Examples of the substituents include hydrocarbyl, aliphatic heterocyclic, aromatic heterocyclic, hydroxyl, alkoxy, aryloxy, aralkyloxy, heteroaryloxy, acyloxy, carbonato, acyl, carboxyl, alkoxycarbonyl, aryloxycarbonyl, aralkyloxycarbonyl, heteroaryloxycarbonyl, carbamoyl, hydroxamic acid, mercapto, alkylthio, arylthio, aralkylthio, heteroarylthio, acylthio, alkoxycarbonylthio, sulfinyl, sulfino, sulfenamoyl, sulfonyl, sulfo, sulfamoyl, amino, hydrazino, ureido, nitro, phosphino, phosphinyl, phosphinico, phosphono, silyl, boryl, and cyano groups, halogen atoms, and the like.

The substituents represented by R^{A}, R^{B}, R^{C}, and R^{D} will be described below in more detail. Examples of the hydrocarbyl groups include alkyl, alkenyl, alkynyl, aryl and aralkyl groups, and the like. Among them, the alkyl group is a straight-chain, branched, or cyclic alkyl group having, for example, 1 to 15 carbon atoms, preferably having 1 to 10 carbon atoms, and more preferably having 1 to 6 carbon atoms; and typical examples thereof include methyl, ethyl, n-propyl, 2-propyl, n-butyl, 2-butyl, isobutyl, tert-butyl, n-pentyl, 2-pentyl, tert-pentyl, 2-methylbutyl, 3-methylbutyl, 2,2-dimethylpropyl, n-hexyl, 2-hexyl, 3-hexyl, tert-hexyl, 2-methylpentyl, 3-methylpentyl, 4-methylpentyl, 2-methyl pentan-3-yl, cyclopropyl, cyclobutyl, cyclopentyl and cyclohexyl groups, and the like. The alkenyl group is a straight-chain or branched alkenyl group having, for example, 2 to 15 carbon atoms, preferably having 2 to 10 carbon atoms, and more preferably having 2 to 6 carbon atoms; and typical examples thereof include ethenyl, propenyl, 1-butenyl, pentenyl and hexenyl groups, and the like. The alkynyl group is a straight-chain or branched alkynyl group having, for example, 2 to 15 carbon atoms, preferably having 2 to 10 carbon atoms, and more preferably having 2 to 6 carbon atoms; and typical examples thereof include ethynyl, 1-propynyl, 2-propynyl, 1-butynyl, 3-butynyl, pentynyl and hexynyl groups, and the like. The aryl group is an aryl group having, for example, 6 to 14 carbon atoms; and typical examples thereof include phenyl, naphthyl, anthryl, phenanthryl, chrysenyl, pyrenyl and biphenyl groups, and the like. The aralkyl group is a group in which at least one hydrogen atom of the above alkyl group is substituted with the aryl group above, preferably an aralkyl group having, for example, 7 to 13 carbon atoms; and typical examples thereof include benzyl, 2-phenylethyl, 1-phenylpropyl and 3-naphthylpropyl groups, and the like.

Examples of the aliphatic heterocyclic group include five- to eight-membered, preferably five- or six-membered, monocyclic aliphatic heterocyclic and polycyclic or fused aliphatic heterocyclic groups, for example, having 2 to 14 carbon atoms and containing at least one, preferably one to three, heteroatom such as nitrogen, oxygen, or sulfur. Typical examples of the aliphatic heterocyclic groups include 2-oxopyrrolidine-1-yl, piperidino, piperadinyl, morpholino, tetrahydrofuryl, tetrahydropyranyl and tetrahydrothienyl groups, and the like.

Examples of the aromatic heterocyclic group include five- to eight-membered, preferably five- or six-membered, monocyclic hetero-aryl and polycyclic or fused hetero-aryl groups having, for example, 2 to 15 carbon atoms and containing at least one, preferably one to three, heteroatom such as nitrogen, oxygen, or sulfur; and typical examples thereof include furyl, thienyl, pyridyl, pyrimidyl, pyradyl, pyridazyl, pyrazolyl, imidazolyl, oxazolyl, thiazolyl, benzofuryl, benzothienyl, quinolyl, isoquinolyl, quinoxalyl, phthalazyl, quinazolyl, naphthylidyl, cinnolyl, benzimidazolyl, benzoxazolyl and benzothiazolyl groups, and the like.

The alkoxy group is a straight-chain, branched, or cyclic alkoxy group having, for example, 1 to 6 carbon atoms; and typical examples thereof include methoxy, ethoxy, n-propoxy, 2-propoxy, n-butoxy, 2-butoxy, isobutoxy, tert-butoxy, n-pentyloxy, 2-methylbutoxy, 3-methylbutoxy, 2,2-dimethylpropyloxy, n-hexyloxy, 2-methylpentyloxy, 3-methylpentyloxy, 4-methylpentyloxy, 5-methylpentyloxy and cyclohexyloxy groups, and the like.

The aryloxy group is an aryloxy group having, for example, 6 to 14 carbon atoms; and typical examples thereof include phenyloxy, naphthyloxy and anthryloxy groups, and the like.

The aralkyloxy group is an aralkyloxy group having, for example, 7 to 12 carbon atoms; and typical examples thereof include benzyloxy, 2-phenylethoxy, 1-phenylpropoxy, 2-phenylpropoxy, 3-phenylpropoxy, 1-phenylbutoxy, 2-phenylbutoxy, 3-phenylbutoxy, 4-phenylbutoxy, 1-phenylpentyloxy, 2-phenylpentyloxy, 3-phenylpentyloxy, 4-phenylpentyloxy, 5-phenylpentyloxy, 1-phenylhexyloxy, 2-phenylhexyloxy, 3-phenylhexyloxy, 4-phenylhexyloxy, 5-phenylhexyloxy and 6-phenylhexyloxy groups, and the like.

The heteroaryloxy group is a heteroaryloxy group, for example, having 2 to 14 carbon atoms and containing at least one, preferably one to three, heteroatom such as nitrogen, oxygen, or sulfur; and typical examples thereof include 2-pyridyloxy, 2-pyrazyloxy, 2-pyrimidynyloxy and 2-quinolyloxy groups, and the like.

The acyloxy group is an acyloxy group having, for example, 2 to 18 carbon atoms and derived from carboxylic acid; and typical examples thereof include acetoxy, propionyloxy, acryloyloxy, butyryloxy, pivaloyloxy, pentanoyloxy, hexanoyloxy, lauroyloxy, stearoyloxy and benzoyloxy groups, and the like.

The alkoxycarbonyloxy group, is a straight-chain, branched, or cyclic alkoxycarbonyloxy group having, for example, 2 to 19 carbon atoms; and typical examples thereof include methoxycarbonyloxy, ethoxycarbonyloxy, n-propoxycarbonyloxy, 2-propoxycarbonyloxy, n-butoxycarbonyloxy, tert-butoxycarbonyloxy, pentyloxycarbonyloxy, hexyloxycarbonyloxy, 2-ethylhexyloxycarbonyloxy, lauryloxycarbonyloxy, stearyloxycarbonyloxy and cyclohexyloxycarbonyloxy groups, and the like.

The acyl group is a straight-chain or branched acyl group having, for example, 1 to 18 carbon atoms and derived from a carboxylic acid such as a fatty carboxylic acid or an aromatic carboxylic acid; and typical examples thereof include formyl, acetyl, propionyl, acryloyl, butyryl, pivaloyl, pentanoyl, hexanoyl, lauroyl, stearoyl and benzoyl groups, and the like.

The alkoxycarbonyl group is a straight-chain, branched, or cyclic alkoxycarbonyl group having, for example, 2 to 19 carbon atoms; and typical examples thereof include methoxycarbonyl, ethoxycarbonyl, n-propoxycarbonyl, 2-propoxycarbonyl, n-butoxycarbonyl, tert-butoxycarbonyl, pentyloxycarbonyl, hexyloxycarbonyl, 2-ethylhexyloxycarbonyl, lauryloxycarbonyl, stearyloxycarbonyl and cyclohexyloxycarbonyl groups, and the like.

The aryloxycarbonyl group is an aryloxycarbonyl group having, for example, 7 to 20 carbon atoms; and typical examples thereof include phenoxycarbonyl and naphthyloxycarbonyl groups, and the like.

The aralkyloxycarbonyl group is an aralkyloxycarbonyl group having, for example, 8 to 15 carbon atoms; and typical examples thereof include benzyloxycarbonyl, phenylethoxycarbonyl and 9-fluorenylmethyloxycarbonyl groups, and the like.

The heteroaryloxycarbonyl group is a heteroaryloxy group having, for example, 3 to 15 carbon atoms and containing at least one, preferably one to three, heteroatom such as a nitrogen, oxygen, or sulfur atom; and typical examples thereof include 2-pyridyloxycarbonyl, 2-pyrazyloxycarbonyl, 2-pyrimidyloxycarbonyl and 2-quinolyloxycarbonyl groups, and the like.

The carbamoyl group is, for example, an unsubstituted carbamoyl group or a carbamoyl group, one or two hydrogen atoms on the nitrogen atom of which are substituted with a substituent group such as the hydrocarbyl group described above; and typical examples thereof include N-methylcarbamoyl, N,N-diethylcarbamoyl and N-phenylcarbamoyl groups, and the like.

The alkylthio group is a straight-chain, branched, or cyclic alkylthio group having, for example, 1 to 6 carbon atoms; and typical examples thereof include methylthio, ethylthio, n-propylthio, 2-propylthio, n-butylthio, 2-butylthio, isobutylthio, tert-butylthio, pentylthio, hexylthio and cyclohexylthio groups, and the like.

The arylthio group is an arylthio group having, for example, 6 to 14 carbon atoms; and typical examples thereof include phenylthio and naphthylthio groups and the like. The aralkylthio group is an aralkylthio group having, for example, 7 to 12 carbon atoms; and typical examples thereof include benzylthio and 2-phenethylthio groups and the like.

The heteroarylthio group is a heteroarylthio group having, for example, 2 to 14 carbon atoms and containing at least one, preferably one to three, heteroatom such as a nitrogen, oxygen, or sulfur atom; and typical examples thereof include 4-pyridylthio, 2-benzimidazolylthio, 2-benzoxazolylthio and 2-benzothiazolylthio groups, and the like.

The acylthio group is an acylthio group having, for example, 2 to 18 carbon atoms and derived from a thiocarboxylic acid; and typical examples thereof include acetylthio, propionylthio, acrylthio, butyrylthio, pivaloylthio, pentanoylthio, hexanoylthio, lauroylthio, stearoylthio and benzoylthio groups, and the like.

The alkoxycarbonylthio group is a straight-chain, branched, or cyclic alkoxycarbonylthio group having, for example, 2 to 19 carbon atoms; and typical examples thereof include methoxycarbonylthio, ethoxycarbonylthio, n-propoxycarbonylthio, 2-propoxycarbonylthio, n-butoxycarbonylthio, tert-butoxycarbonylthio, pentyloxycarbonylthio, hexyloxycarbonylthio, 2-ethylhexyloxycarbonylthio, lauryloxycarbonylthio, stearyloxycarbonylthio and cyclohexyloxycarbonylthio groups, and the like.

The sulfinyl group is, for example, a sulfinyl group, of which the hydrogen atom on the sulfur atom is substituted with a substituent such as the hydrocarbyl group described above; and typical examples thereof include methanesulfinyl, benzenesulfinyl and p-toluenesulfinyl groups, and the like.

The sulfenamoyl group is, for example, an unsubstituted sulfenamoyl group or a sulfenamoyl group, of which the hydrogen atom on the nitrogen atom is substituted with a substituent such as the hydrocarbyl group described above; and typical examples thereof include N-methylsulfenamoyl, N,N-diethylsulfenamoyl and N-phenylsulfenamoyl groups, and the like.

The sulfonyl group is, for example, a sulfonyl group, of which the hydrogen atom on the sulfur atom is substituted with a substituent such as the hydrocarbyl group described above; and typical examples thereof include methanesulfonyl, benzenesulfonyl and p-toluenesulfonyl groups, and the like.

The sulfamoyl group is, for example, an unsubstituted sulfamoyl group or a sulfamoyl group, of which the hydrogen atom on the nitrogen atom is substituted with a substituent such as the hydrocarbyl group described above; and typical examples thereof include N-methylsulfamoyl, N,N-diethylsulfamoyl and N-phenylsulfamoyl groups, and the like.

The amino group is, for example, an unsubstituted amino group or an amino group, of which the hydrogen atom on the nitrogen atom is substituted with a substituent such as an amino-protecting group. For example, any one of the protecting groups described above may be used as the amino-protecting group, and typical examples thereof include the alkyl, aryl, aralkyl, acyl, alkoxycarbonyl, aryloxycarbonyl, aralkyloxycarbonyl and sulfonyl groups described above, and the like.

Typical examples of the alkyl group-substituted amino group, i.e., alkylamino group, include mono- or di-alkylamino groups such as N-methylamino, N,N-dimethylamino, N,N-diethylamino, N,N-diisopropylamino and N-cyclohexylamino groups, and the like.

Typical examples of the aryl group-substituted amino group, i.e., arylamino group include mono- or di-arylamino groups such as N-phenylamino, N,N-diphenylamino, N-naphthylamino and N-naphthyl-N-phenylamino groups.

Typical examples of the aralkyl group-substituted amino group, i.e., aralkylamino group, include mono- or di-aralkylamino groups such as N-benzylamino and N,N-dibenzylamino groups.

Typical examples of the acyl group-substituted amino group, i.e., acylamino group, include formylamino, acetylamino, propionylamino, acryloylamino, pivaloylamino, pentanoylamino, hexanoylamino and benzoylamino groups, and the like.

Typical examples of the alkoxycarbonyl group-substituted amino group, i.e., alkoxycarbonylaminogroup, include methoxycarbonylamino, ethoxycarbonylamino, n-propoxycarbonylamino, n-butoxycarbonylamino, tert-butoxycarbonylamino, pentyloxycarbonylamino and hexyloxycarbonylamino groups, and the like.

Typical examples of the aryloxycarbonyl group-substituted amino group, i.e., aryloxycarbonylamino group, include phenoxycarbonylamino and naphthyloxycarbonylamino groups, and the like.

Typical examples of the aralkyloxycarbonyl group-substituted amino group, i.e., aralkyloxycarbonylamino group include a benzyloxycarbonylamino group and the like.

Typical examples of the sulfonyl group-substituted amino group, i.e., sulfonylamino group, include methanesulfonylamino and p-toluenesulfonylamino groups, and the like.

The hydrazino group is, for example, an unsubstituted hydrazino group and a hydrazino group, of which at least one hydrogen atom on the nitrogen atom is substituted with a substituent such as the hydrocarbyl group described above; and typical examples thereof include 2-methylhydrazino, 2,2-dimethylhydrazino, 1,2,2-trimethylhydrazino, 2-phenylhydrazino and 2,2-diphenylhydrazino groups, and the like.

The ureido group is, for example, an unsubstituted ureido group or an ureido group, of which at least one hydrogen atom on the nitrogen atom is substituted with a substituent such as the hydrocarbyl group described above; and typical examples thereof include 3-methylureido, 1,3,3-trimethylureido and 3,3-diphenylureido groups, and the like.

The phosphino group is, for example, a phosphino group, of which two hydrogen atoms on the phosphorus atom are substituted with a substituent such as the hydrocarbyl group described above; and typical examples thereof include dimethylphosphino, diphenylphosphino, di (2-furyl)phosphino and dibenzylphosphino groups, and the like.

The phosphinyl group is, for example, a phosphinyl group, of which two hydrogen atoms on the phosphorus atom are substituted with a substituent such as the hydrocarbyl group described above; and typical examples thereof include dimethylphosphinyl and diphenylphosphinyl groups, and the like.

The phosphinico group is, for example, an unsubstituted phosphinico group or a phosphinico group, of which the hydrogen atom on the oxygen atom is substituted with a substituent such as the hydrocarbyl group described above; and typical examples thereof include methylphosphinico, ethylphosphinico, phenylphosphinico and benzylphosphinico groups, and the like.

The phosphono group is, for example, an unsubstituted phosphono group or a phosphono group, of which the hydrogen atom on the oxygen atom is substituted with a substituent such as the hydrocarbyl group described above; and typical examples thereof include dimethylphosphono, diethylphosphono, phenylphosphono, diphenylphosphono and dibenzylphosphono groups, and the like.

The silyl group is, for example, a silyl group, of which the hydrogen atom on the silicon atom is substituted with a substituent such as the hydrocarbyl group described above; and typical examples thereof include trimethylsilyl, triisopropylsilyl, tert-butyldimethylsilyl, tert-butyldiphenylsilyl and triphenylsilyl groups, and the like.

The boryl group is, for example, a boryl group, of which the two hydrogen atoms on the boron atom are substituted with a substituent such as the hydrocarbyl group described above; and typical examples thereof include a bis(2,4,6-trimethylphenyl)boryl group and the like.

Examples of the halogen atoms include fluorine, chlorine, bromine and iodine atoms, and the like.

When there are two or more substituents on the same ring, these substituents may be bound each other independently to form a fused ring. In addition, when neighboring rings have respectively one or more substituents, these substituents may be bound each other independently to form a fused ring.

The luminescence maximum wave (λmax) of the phosphorescence obtained from the platinum complex of the present invention is 350 nm to 520 nm, preferred 400 nm to 500 nm, more preferred 420 nm to 500 nm when said platinum complex was used in the light (green to blue) emission device.

Hereinafter, typical examples of the platinum complex represented by General Formula (1) of the present invention are listed below, but the present invention is not restricted thereby.

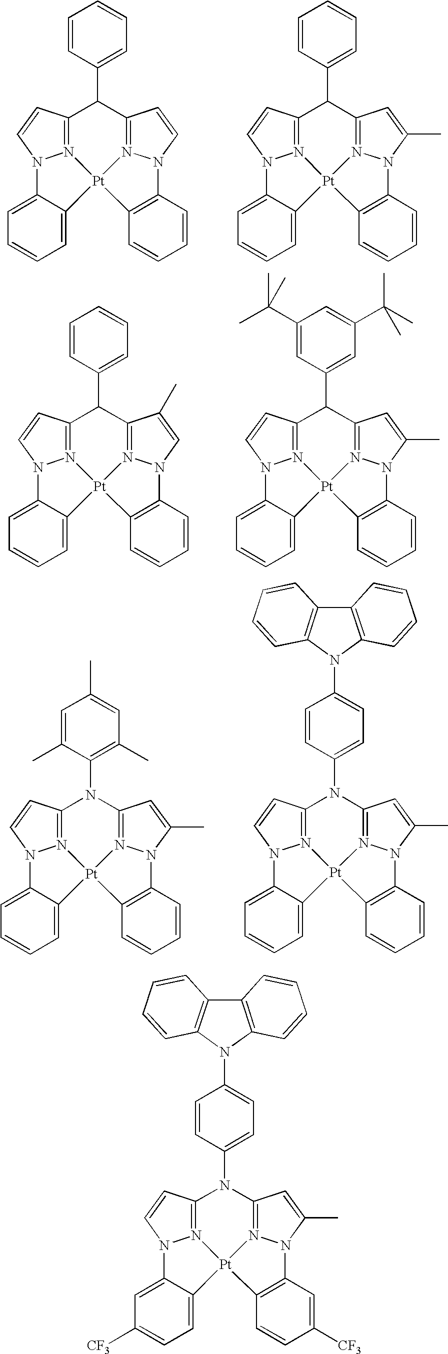

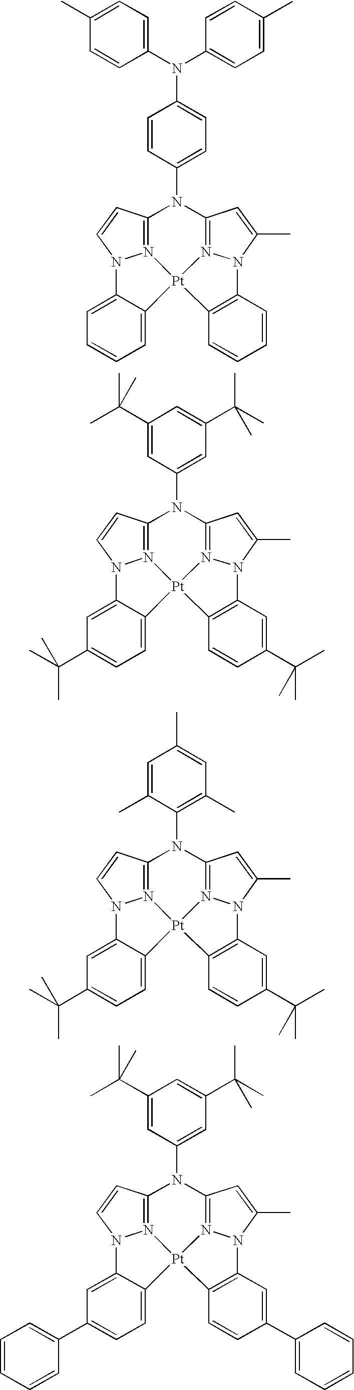

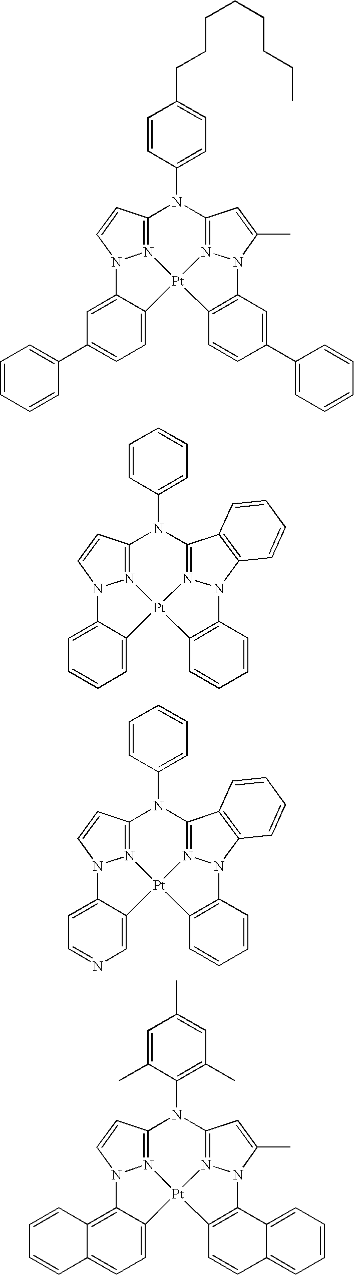

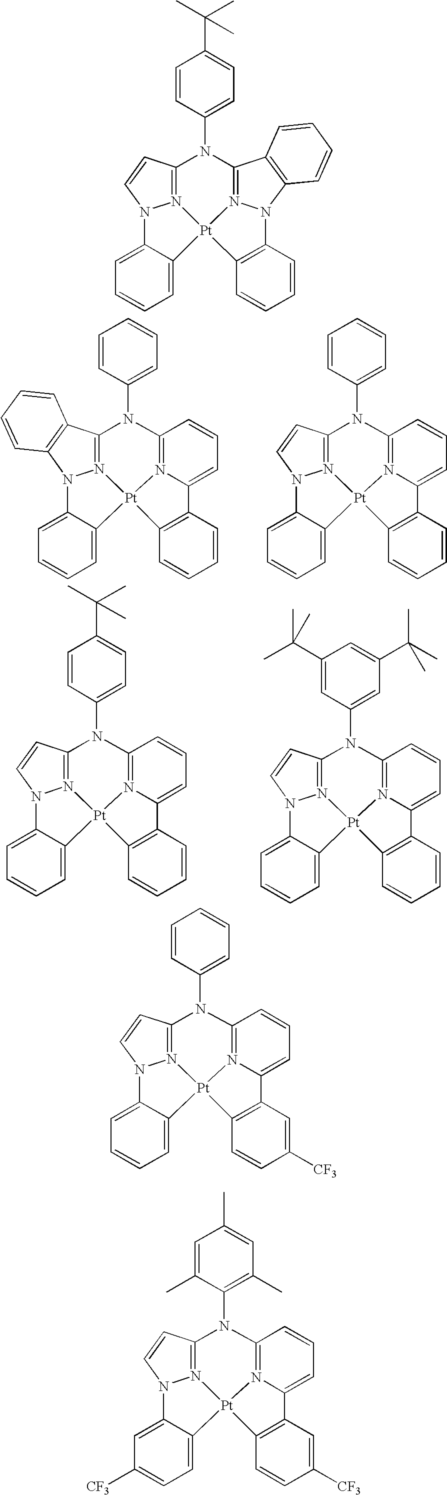

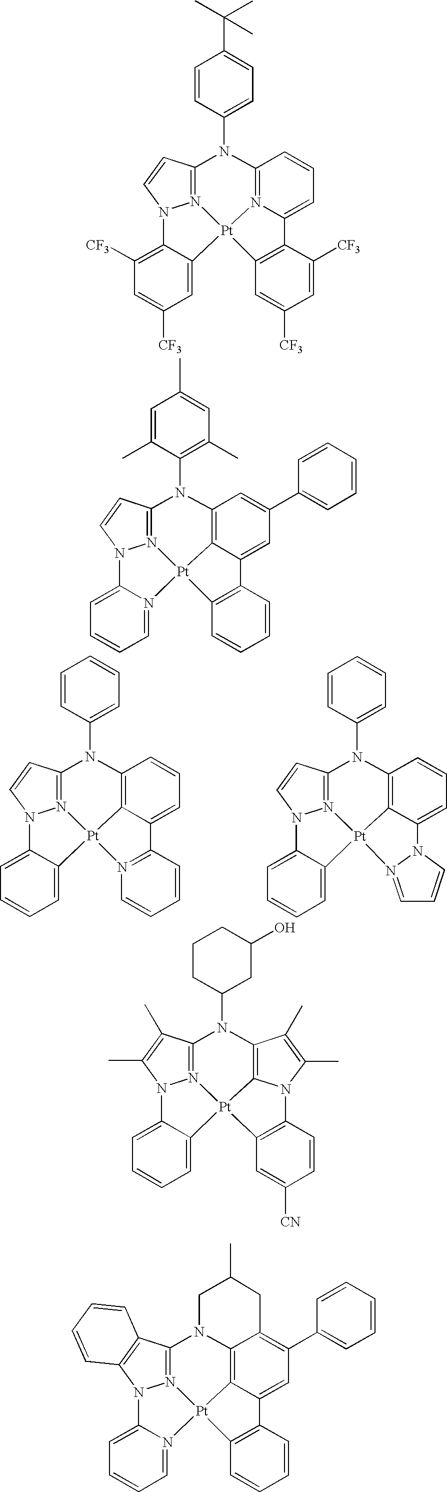

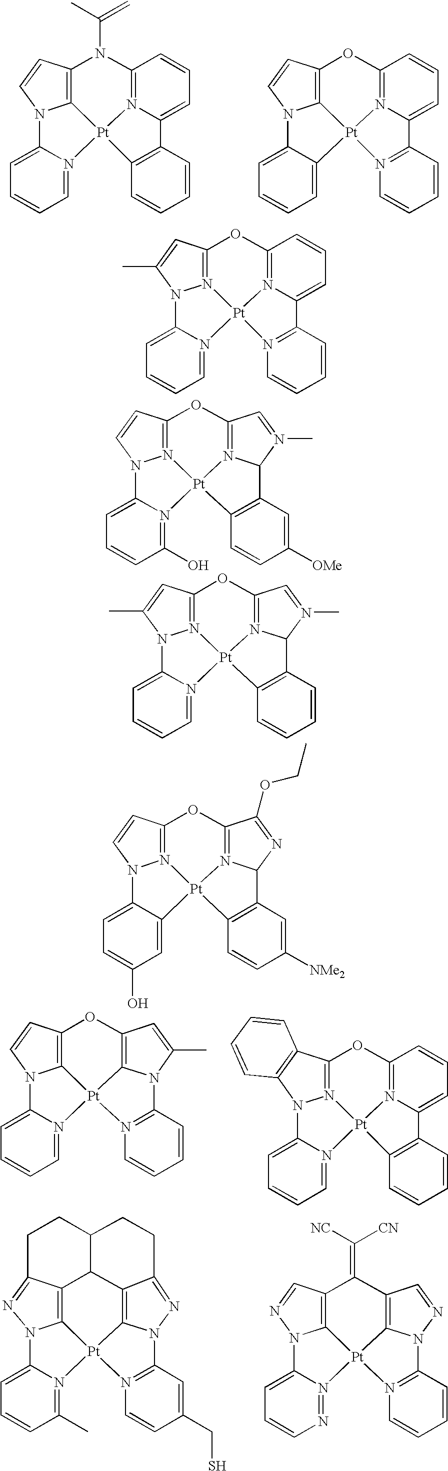

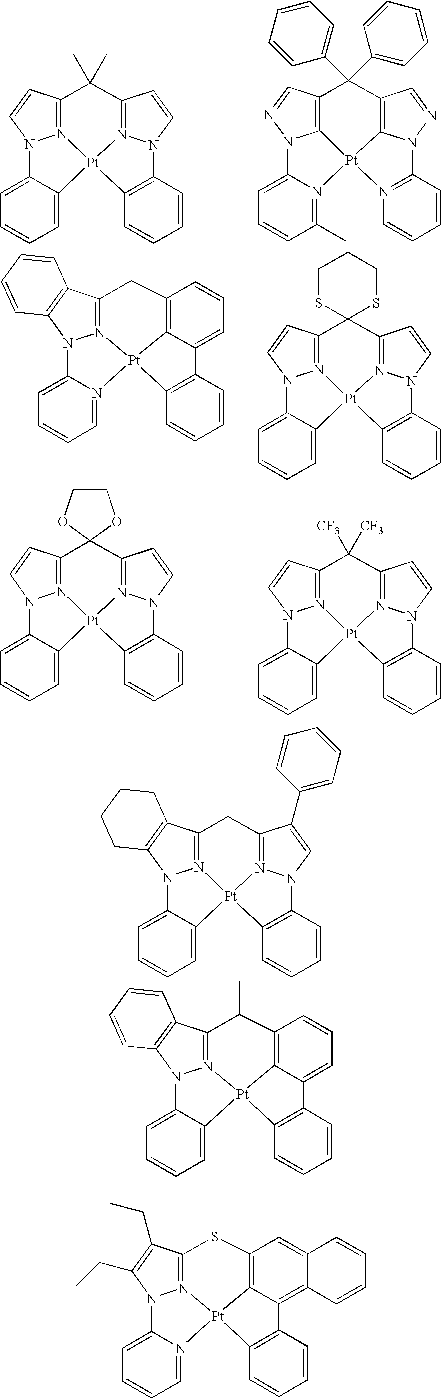

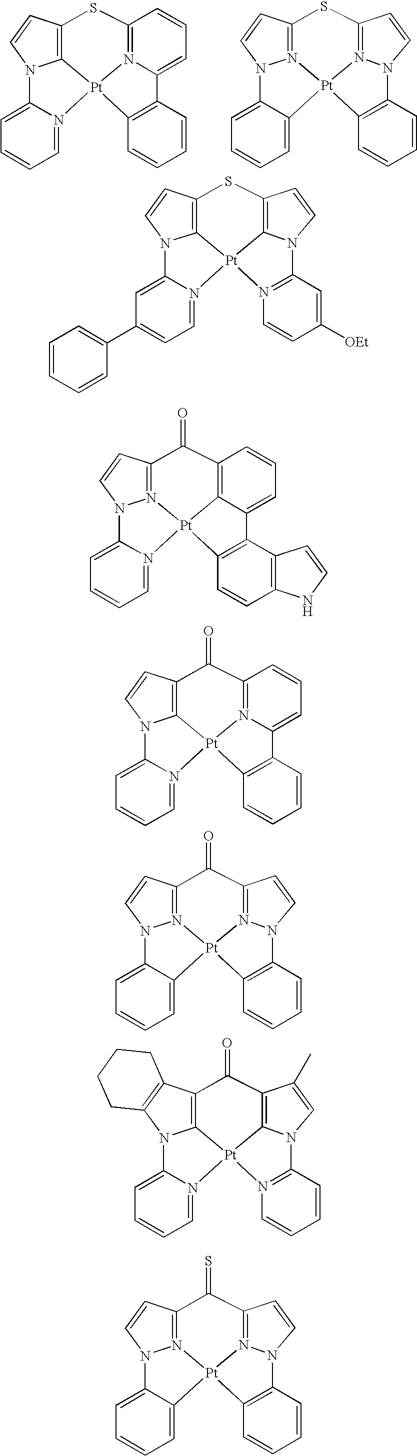

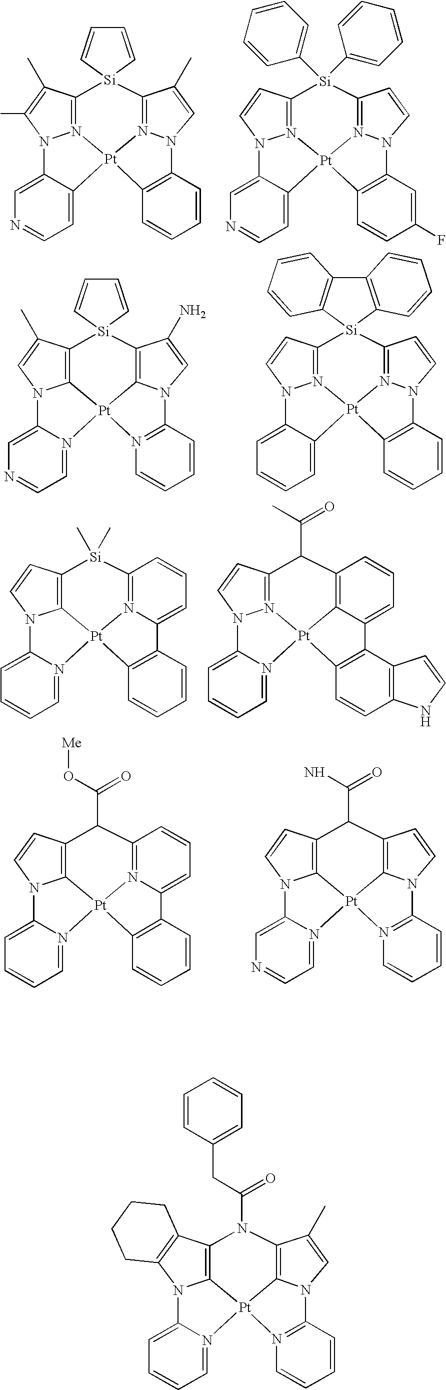

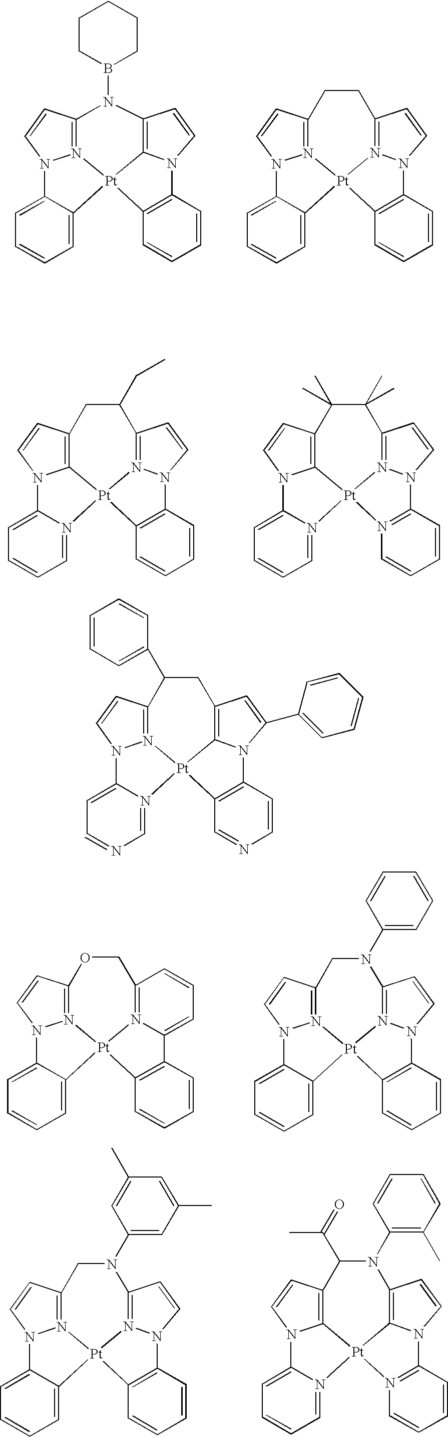

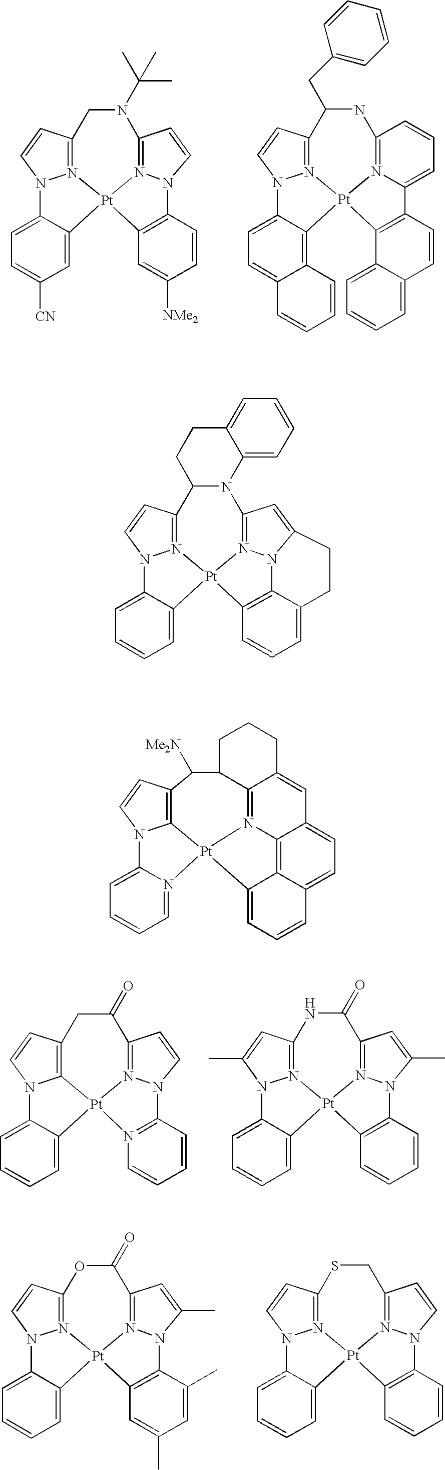

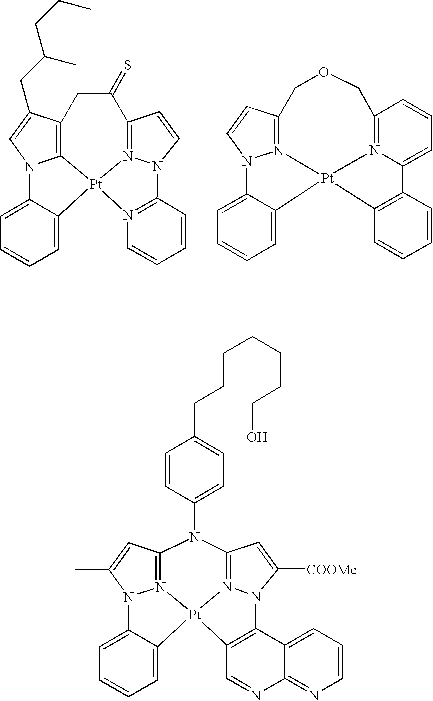

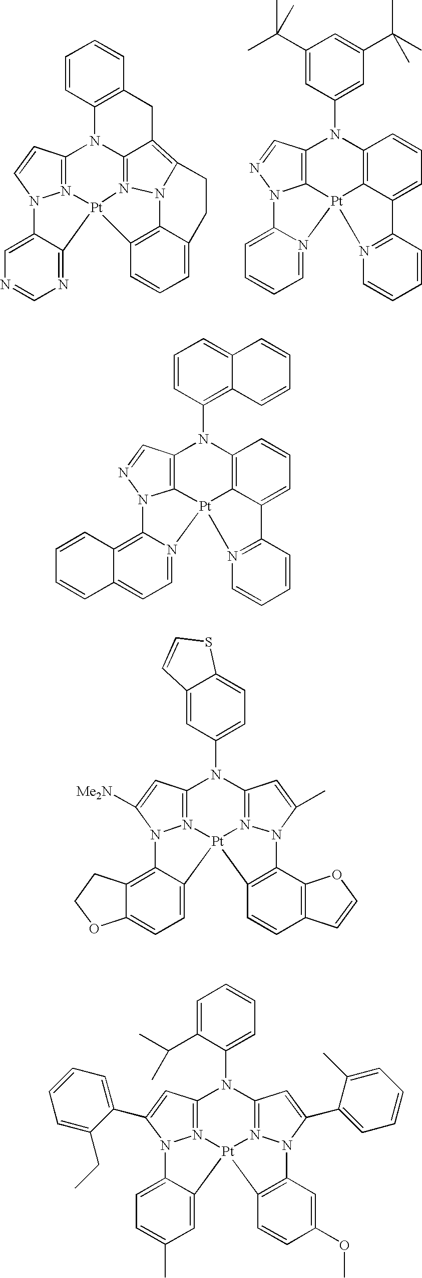

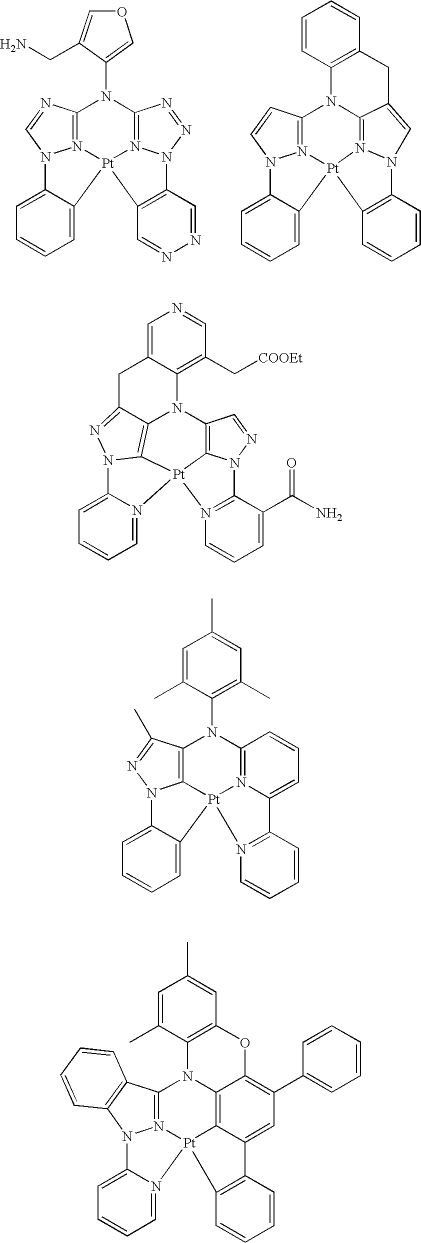

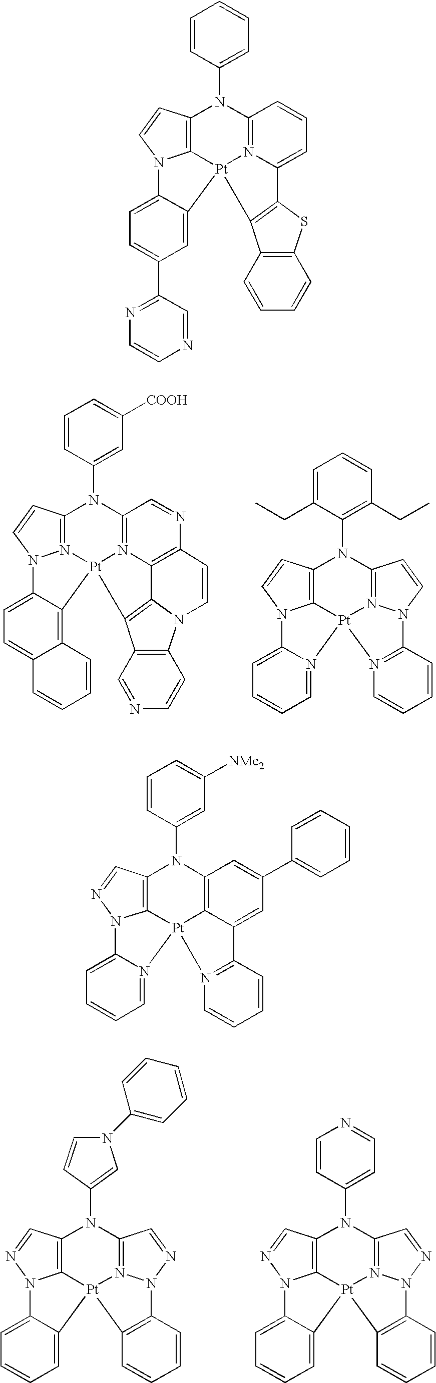

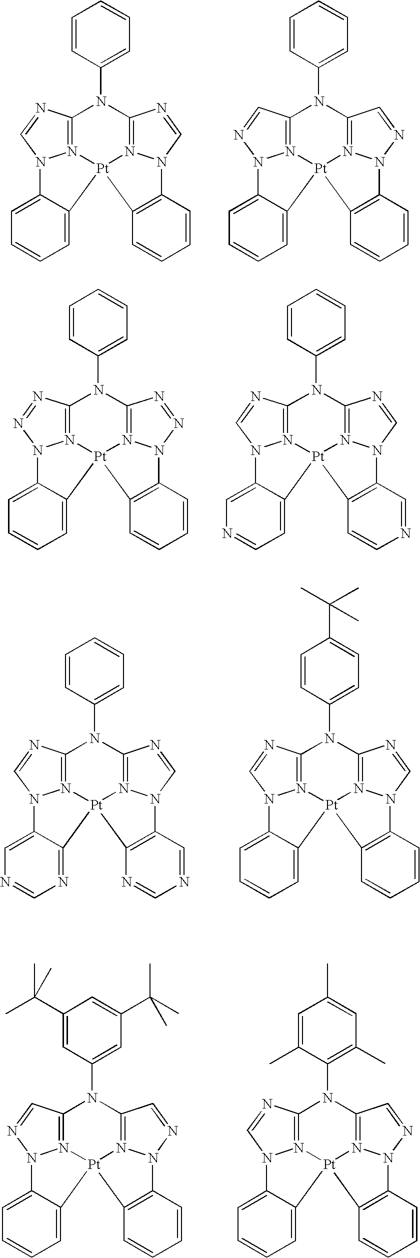

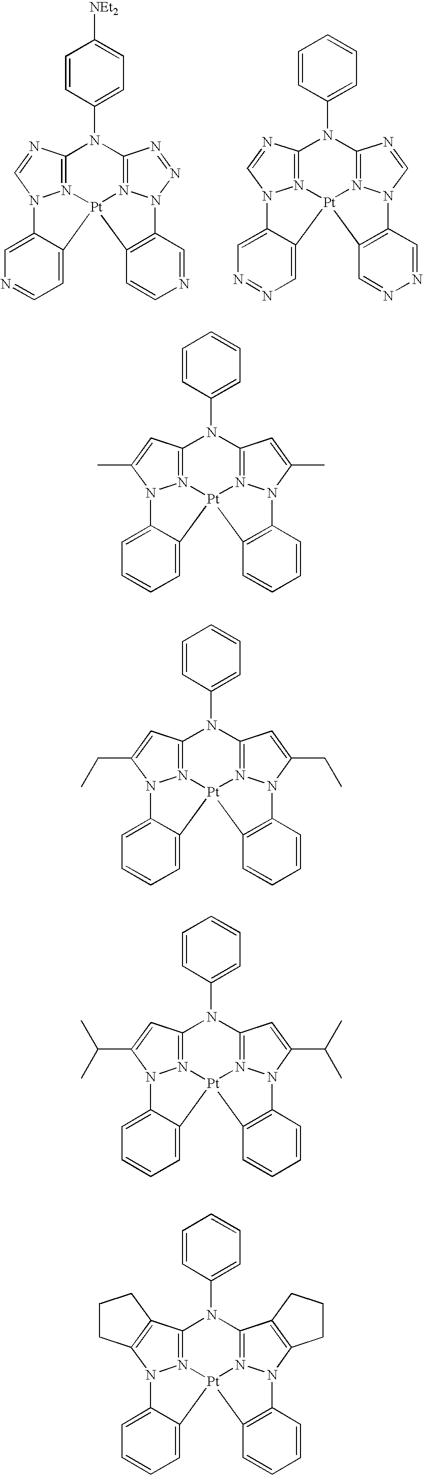

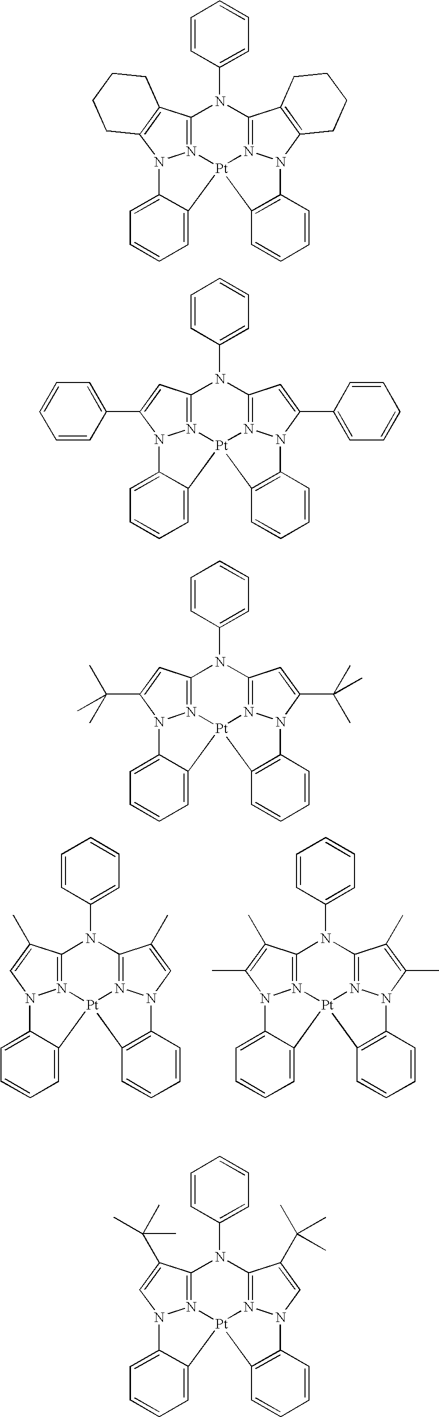

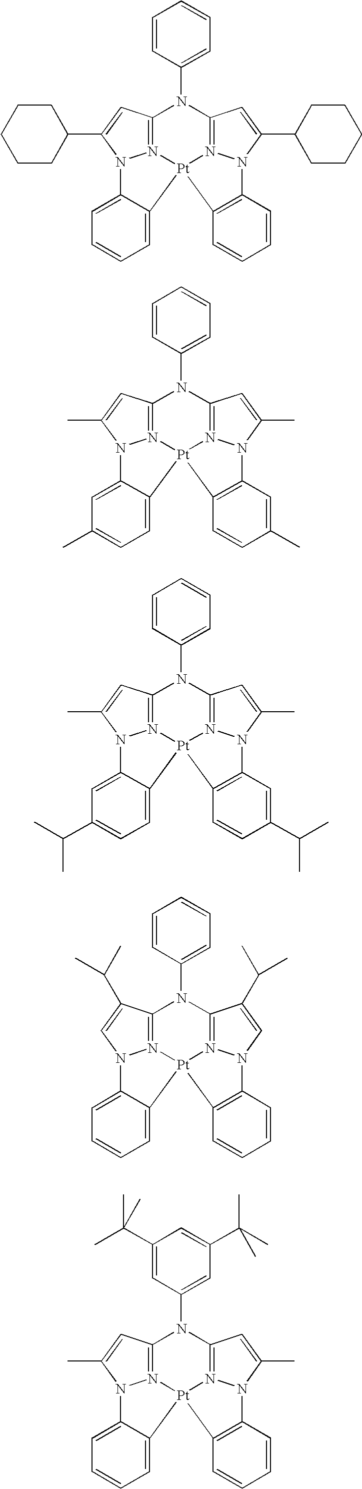

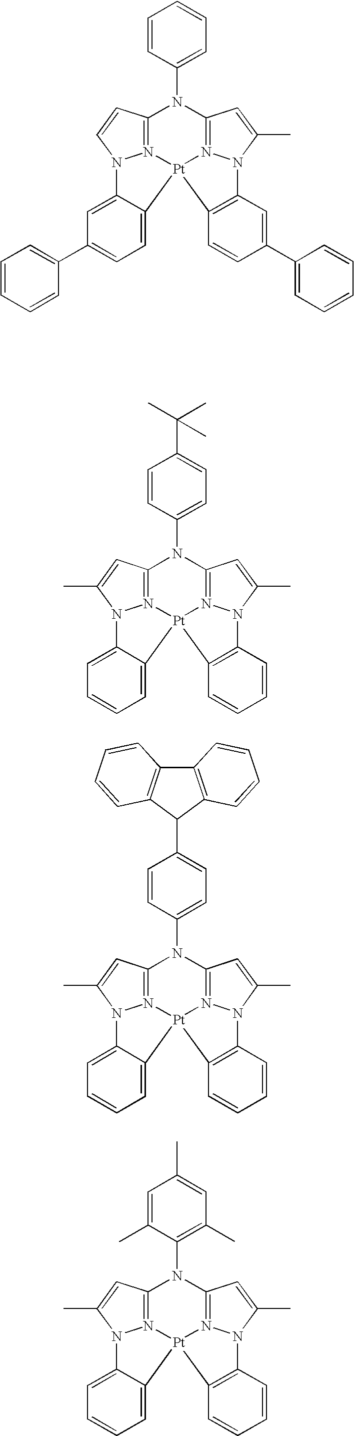

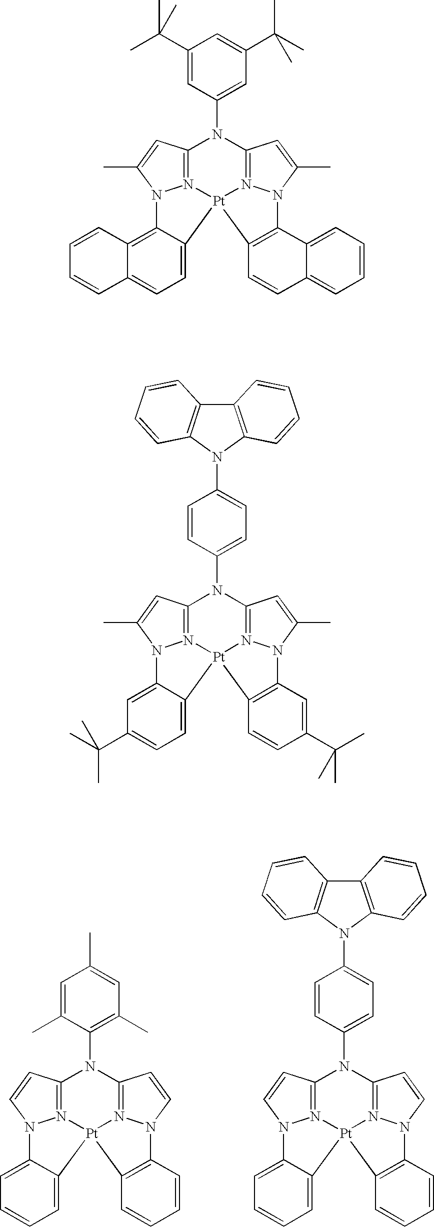

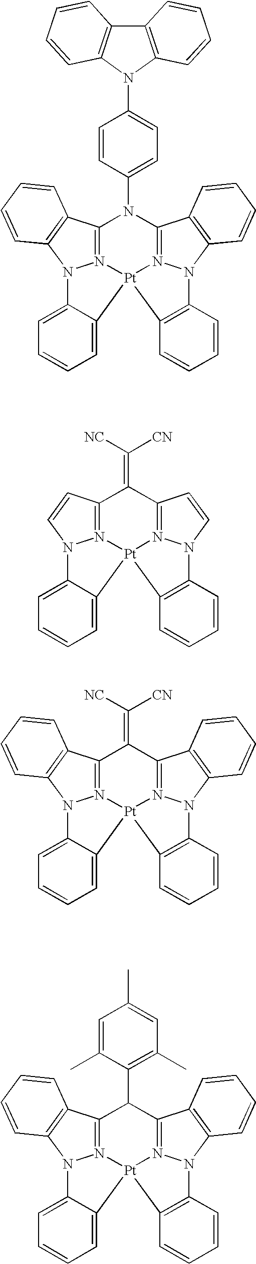

Hereinafter, the method of producing the platinum complex of the present invention will be described.

The compound represented by General Formula (1) can be prepared easily in the reaction of a platinum complex precursor with the compound represented by General Formula (2) as shown in the following Scheme 1:

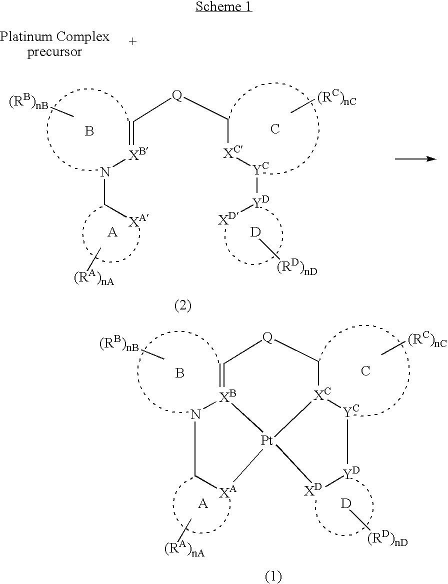

wherein the compounds represented by General Formulae (1) and (2) are the same as those described above.

Hereinafter, the compound represented by General Formula (2) will be referred to simply as “the tetradentate ligand of the present invention”.

Both inorganic and organic platinum complexes may be used favorably as the platinum complex precursor used in the production method according to the present invention. Favorable examples of the inorganic platinum compounds include platinum halides such as platinum chloride, platinum bromide and platinum iodide; and haloplatinic acid salts such as sodium chloroplatinate, potassium chloroplatinate, potassium bromoplatinate and potassium iodoplatinate. Platinum chloride and potassium chloroplatinate are used more favorably, because of the easiness in procurement.

The organic platinum complex is preferably an organic platinum complex having a monodentate or bidentate ligand from a viewpoint of chelating effect. Typical examples thereof include platinum olefin complexes such as di-μ-chloro-dichloroethylenediplatinum, dichloro(η-1,5-hexadiene)platinum, dichloro(η-1,5-cyclooctadiene)platinum, (η-bicyclo[2,2,1]hepta-2,5-diene)dichloroplatinum and bis(η-1,5-cyclooctadiene)platinum; platinum amine complexes such as cis-/trans-bis(ammine)dichloroplatinum and dichloro(ethylenediammine)platinum; platinum nitrogen-containing heterocyclic ring complexes such as cis-/trans-bis(pyridinato)dichloroplatinum and (2,2′-bipyridinato)dichloroplatinum; platinum nitrile complexes such as cis-bis(benzonitrile)dichloroplatinum and cis-/trans-bis(acetonitrile)dichloroplatinum; platinum phosphine complexes such as cis-/trans-bis(tributylphosphine) dichloroplatinum, cis-/trans-bis(triphenylphosphine)dichloroplatinum, dichloro[ethanebis(diphenylphosphine)]platinum and tetrakis(triphenylphosphine)platinum; platinum sulfur-containing compound complexes such as cis-bis(tetrahydrothiophene)dichloroplatinum; and the like.

More preferable examples of the organic platinum complexes include platinum olefin complexes such as dichloro(η-1,5-hexadiene)platinum and dichloro(η-1,5-cyclooctadiene)platinum; platinum nitrile complex such as cis-bis(benzonitrile)dichloroplatinum and cis-/trans-bis(acetonitrile)dichloroplatinum; and the like.

The organic platinum complexes above may be used in the complexation after preparation and isolation, or alternatively, in the so-called one-pot reaction thereof with the tetradentate ligand of the present invention, without isolation after preparation from an inorganic platinum compound. Specifically, cis-bis(benzonitrile) dichloroplatinum, for example, is prepared from platinum chloride and benzonitrile in a system; then a tetradentate ligand of the present invention and other additives if needed are added thereto; and the mixture is allowed to react in the benzonitrile solvent.

The amount of the tetradentate ligand of the present invention used is normally 0.5 to 20 equivalents, preferably 0.8 to 10 equivalents, and more preferably 1.0 to 2.0 equivalents to the amount of the platinum complex precursor.

The platinum complex may be prepared in the absence of a solvent, and is preferably prepared in the presence of a solvent. Typical examples of the preferable solvents include aliphatic hydrocarbons such as pentane, hexane, heptane, octane, decane, dodecane, undecane, cyclohexane and decalin; halogenated aliphatic hydrocarbons such as dichloromethane, 1,2-dichloroethane, chloroform and carbon tetrachloride; aromatic hydrocarbons such as benzene, toluene, xylene, mesitylene, p-cymene and diisopropylbenzene; halogenated aromatic hydrocarbons such as chlorobenzene and o-dichlorobenzene; alcohols such as methanol, ethanol, 2-propanol, n-butanol and 2-ethoxyethanol; polyvalent alcohols such as ethylene glycol, propylene glycol, 1,2-propanediol and glycerol; ethers such as diethyl ether, diisopropyl ether, tert-butyl methyl ether, cyclopentyl methyl ether, dimethoxy ethane, ethylene glycol diethyl ether, tetrahydrofuran and 1,4-dioxane; carboxylic acids such as acetic acid and propionic acid; esters such as methyl acetate, ethyl acetate, n-butyl acetate and methyl propionate; ketones such as acetone, methyl ethyl ketone, methyl isobutyl ketone and cyclohexanone; amines such as triethylamine, aniline and phenethylamine; amides such as formamide, N,N-dimethylformamide and N,N-dimethylacetamide; nitriles such as acetonitrile, malononitrile and benzonitrile; sulfoxides such as dimethyl sulfoxide; water; and the like. These solvents may be used alone or in combination of two or more thereof if needed.

Typical examples of more preferable solvents include aliphatic hydrocarbons such as decane, dodecane, undecane and decalin; aromatic hydrocarbons such as toluene, xylene, mesitylene, p-cymene and diisopropylbenzene; alcohols such as n-butanol and 2-ethoxyethanol; polyvalent alcohols such as ethylene glycol, propylene glycol, 1,2-propanediol and glycerol; ethers such as ethylene glycol diethyl ether, tetrahydrofuran and 1,4-dioxane; carboxylic acids such as acetic acid and propionic acid; esters such as n-butyl acetate and methyl propionate; amides such as N,N-dimethylformamide and N,N-dimethylacetamide; nitriles such as benzonitrile; sulfoxides such as dimethyl sulfoxide; water; and the like. These solvents may be used alone or in combination of two or more thereof if needed.

The amount of the solvent used is not particularly limited, if the reaction proceeds sufficiently, and is properly selected in the range of larger by 1 to 500 times, preferably by 5 to 200 times, and more preferably by 10 to 100 times by volume to the amount of the platinum complex precursor.

The platinum complex may be prepared in the presence of additives added as needed. One of the favorable additives is a base. The bases include, for example, inorganic and organic bases. Favorable examples of the inorganic bases include alkali metal hydroxides such as lithium hydroxide, sodium hydroxide and potassium hydroxide; alkali metal carbonate salts such as lithium carbonate, sodium carbonate and potassium carbonate; alkali metal bicarbonates such as sodium bicarbonate and potassium bicarbonate; and metal hydrides such as sodium hydride. Favorable examples of the organic base include alkali metal alkoxides such as lithium methoxide, sodium methoxide, potassium methoxide, sodium ethoxide, potassium ethoxide, sodium tert-butoxide and potassium tert-butoxide; amines such as triethylamine, diisopropylethylamine, N,N-dimethylaniline, piperidine, pyridine, 4-dimethylaminopyridine, 1,5-diazabicyclo[4.3.0]nona-5-ene, 1,8-diazabicyclo[5.4.0]undeca-7-ene, tri-n-butylamine and N-methylmorpholine; organic alkali metal compounds such as n-butyllithium, tert-butyllithium and phenyllithium; Grignard reagents such as butylmagnesium chloride, phenylmagnesium bromide and methylmagnesium iodide; and the like.

When a base is used as an additive, the amount thereof is properly selected in the range of normally 1 to 10 equivalents, preferably 1.5 to 5 equivalents, and more preferably 2 to 3 equivalents to the amount of the tetradentate ligand.

The compound of the present invention is preferably produced under inert gas atmosphere. Examples of the inert gas include nitrogen and argon gases, and the like. In addition, the platinum complex is also prepared favorably by using an ultrasonic generator or a microwave generator additionally.

The reaction temperature is properly selected in the range of normally 25 to 300° C., preferably 80 to 250° C., and more preferably 120 to 200° C.

The reaction time may vary according to the reaction conditions such as reaction temperature, solvent, and additives, and is selected in the range of normally 10 minutes to 72 hours, preferably 30 minutes to 48 hours, and more preferably 1 to 12 hours.

The platinum complex of the present invention thus obtained may be post-treated, isolated, and purified as needed. The post-treatment methods include, for example, extraction of reaction product, filtration of precipitate, crystallization by addition of solvent, distillation of solvent, and the like; and these post-treatment methods may be used alone or in combination thereof. Examples of the isolation and purification methods include column chromatography, recrystallization, sublimation, and the like; and they may be used alone or in combination thereof.

The tetradentate ligand of the present invention can be prepared in suitable combination of the synthetic reactions such as carbon/carbon bond-forming reactions by using a palladium catalyst such as Suzuki coupling, Negishi coupling, Sonogashira coupling and Stille coupling; carbon/carbon bond-forming reactions by using a nickel catalyst such as Kumada coupling; carbon/nitrogen bond- and carbon/oxygen bond-forming reactions by using a palladium catalyst; carbon/nitrogen bond- and carbon/oxygen bond-forming reactions by using a copper catalyst such as Ullmann coupling; aromatic ring- and aromatic heterocyclic ring-forming reactions by using a cobalt catalyst; aliphatic heterocyclic ring- and aromatic heterocyclic ring-forming reactions by condensation of a nitrogen-containing compound; halogenation reactions by using, for example, bromine, 1,1,2,2-tetrafluoro-1,2-dibromoethane, N-bromosuccinimide or tetrabutylammonium tribromide; Sandmeyer reactions by using a diazonium salt; lithiation reactions by using an alkyllithium or lithium amide reagent; nucleophilic addition/addition elimination reactions by using an organic lithium reagent or a Grignard reagent; electrophilic aromatic substitution reactions such as Friedel-Crafts reaction; quantitative/catalytic oxidation reactions; quantitative/catalytic reductive reactions; and transfer reactions such as sigmatropic transfer. The tetradentate ligand of the present invention also has a characteristic that it is possible to prepare various kinds of derivatives according to the combination of the reagents and reactions used.

Hereinafter, the light-emitting device of the present invention will be described in detail.

The light-emitting device of the present invention is characterized by that at least one platinum complex of the present invention is contained therein. It is not particularly limited by the system, driving method, and application of the light-emitting device of the present invention are not limited as long as the platinum complex of the present invention is used in the device, and a light-emitting device utilizing the emission from the platinum complex above or utilizing the platinum complex above as a charge-transporting material is preferred. A typical example of such a light-emitting device is an organic electroluminescent device (organic EL device).

The light-emitting device of the present invention may be any light-emitting device as long as it is one containing at least one platinum complex of the present invention. When the light-emitting device is prepared by forming a light-emitting layer or multiple organic compound layers including a light-emitting layer between a pair of electrodes, the light-emitting device is characterized by that at least one of the platinum complexes above is contained in at least one layer thereof. The platinum complexes may be contained in combination of two or more thereof as needed.

The method of forming an organic compound layer in the light-emitting device of the present invention is not particularly limited. Examples thereof include methods such as a resistance-heating vapor deposition method, an electron beam method, a sputtering method, a molecular lamination method, a coating method and an inkjet method. Of these, the resistance-heating vapor deposition, coating, and inkjet methods are preferred from viewpoints of properties and productivity of the layer.

The light-emitting device of the present invention is preferably an organic electroluminescent device having a light-emitting layer or multiple organic compound layers including a light-emitting layer between a pair of electrodes, anode and cathode. Examples of the organic compound layers include, in addition to the light-emitting layer, a hole injection layer, a hole transporting layer, an electron injection layer, an electron transporting layer, a protecting layer, and the like; and each of these layers may have other functions as well. Various materials can be used in forming each layer. Hereinafter, each layer will be described in more detail.

The anode supplies holes to the hole injection layer, the hole transporting layer, the light-emitting layers, and the like. And the anode is made of a material such as a metal, an alloy, a metal oxide, an electrically conductive compound, or the mixture thereof. As the material, a material having a work function of 4 eV or more is preferred. Typical examples of the material include electrically conductive metal oxides such as tin oxide, zinc oxide, indium oxide and indium tin oxide (hereinafter, referred to as ITO), metals such as gold, silver, chromium and nickel, mixtures or laminates of the metal above and the electrically conductive metal oxide, inorganic conductive substances such as copper iodide and copper sulfide, organic conductive substances such as polyaniline, polythiophene and polypyrrole, lamination layers of an inorganic/organic conductive substance and ITO, and the like. Of these, electrically conductive metal oxides are preferred, and ITO is particularly preferable from viewpoints, for example, of productivity, high conductivity and transparency.

The thickness of the anode is properly decided according to the material used, and is selected in the range of preferably 10 nm to 5 μm, more preferably 20 nm to 1 μm, and still more preferably 30 nm to 500 nm. The anode used is normally formed as a layer on a material such as soda lime glass, nonalkali glass or transparent resin substrate. When a glass is used, use of a nonalkali glass as the anode substrate is preferable, because of reducing the amount of ions eluted from the glass. Alternatively, soda lime glass, if used, is preferably barrier-coated, for example, with silica. The thickness of the substrate is not particularly limited if it is sufficient for preserving a desirable mechanical strength, and is normally 0.2 mm or more, preferably 0.7 mm or more, when a glass is used. Various methods may be used for preparation of the anode. When ITO is used as an anode material, the ITO anode layer is formed by a method such as an electron beam method, a sputtering method, a resistance-heating vapor deposition method, a chemical reaction method, or a coating method. Reduction of the drive voltage and improvement in the luminous efficiency of the device may be achieved by the cleaning or other processing of the anode. For example, UV-ozone treatment, plasma treatment, and the like are effective in processing ITO anodes. It is preferable that the sheet resistance of the anode is lower.

On the other hand, the cathode supplies electrons to the electron injection layer, the electron transporting layer, the light-emitting layer, and the like; and is selected, considering the adhesiveness to the layer next to the cathode such as an electron injection layer, an electron transporting layer or a light-emitting layer, the ionization potential, and the stability thereof. As the material for the cathode, a metal, an alloy, a metal halide, a metal oxide, an electrically conductive compound, or the mixture thereof may be used; and typical examples of the materials include alkali metals such as lithium, sodium and potassium and the fluorides thereof, alkali-earth metals such as magnesium and calcium and the fluorides thereof, metals such as gold, silver, lead, aluminum and indium, rare earth metals such as ytterbium, mixed metals such as sodium-potassium alloy, lithium-aluminum alloy and magnesium-silver alloy, and the like. As the material, a material having a work function of 4 eV or more is preferred, and examples of more preferable materials include aluminum, an alloy of lithium and aluminum, an alloy of magnesium and silver, the mixed metal thereof, or the like. The cathode may have a lamination structure containing therein the compound above or the mixture thereof.

The thickness of the cathode may be selected properly according to the material used, and is selected in the range of preferably 10 nm to 5 μm, more preferably 50 nm to 1 μm, and still more preferably 100 nm to 1 μm. The cathode is formed by a method such as an electron beam method, a sputtering method, a resistance-heating vapor deposition method, or a coating method; and a single metal deposition or two or more component simultaneous deposition method may be used in the vapor deposition. Alternatively, an alloy cathode can be formed by simultaneous vapor deposition of multiple metals, or alternatively, by vapor deposition of an alloy previously prepared. It is preferable that the sheet resistance of the cathode is lower.

The material for the light-emitting layer is not particularly limited, if it can form a layer having a function of receiving holes from the anode, the hole injection layer or the hole transporting layer, a function of receiving electrons from the cathode, the electron injection layer, the electron transporting layer or the hole blocking layer and a function of providing a site for recombination of the holes and the electrons to emit light, when an electric field is applied. Typical examples of thereof include carbazole derivatives, arylamine derivatives, styrylamine derivatives, benzoxazole derivatives, benzothiazole derivatives, benzimidazole derivatives, oxadiazole derivatives, coumarin derivatives, perynone derivatives, naphthalimide derivatives, aldazine derivatives, quinacridone derivatives, pyrrolopyridine derivatives, thiadiazopyridine derivatives, oligophenylene derivatives, styrylbenzene derivatives, diphenylbutadiene derivatives, tetraphenylbutadiene derivatives, bisstyrylanthracene derivatives, perylene derivatives, cyclopentadiene derivatives, aromatic dimethylidene compounds, arylborane derivatives, arylsilane derivatives, various typical, transition or rare-earth metal complexes including metal complexes with an 8-quinolinol derivative as the ligand, polymer or oligomer compounds such as poly(N-vinylcarbazole), polythiophene, polyphenylene, and polyphenylene vinylene, the tetradentate ligands of the present invention, the platinum complexes of the present invention, and the like. Each of the polymer or oligomer compounds may have the tetradentate ligand of the present invention or the platinum complex of the present invention as its partial structure independently. The materials for the light-emitting layer are not limited to the typical examples exemplified above.

The light-emitting layer may have a single-layered structure containing one or more of the materials above or a multilayer structure having multiple layers same or different in composition. The thickness of the light-emitting layer is not particularly limited, and is selected in the range of preferably 1 nm to 5 μm, more preferably 5 nm to 1 μm, and still more preferably 10 to 500 nm. The method of forming the light-emitting layer is not particularly limited, and examples thereof include an electron beam method, a sputtering method, a resistance-heating vapor deposition method, a molecular lamination method, a coating method, an inkjet method, and a LB method; and preferred are the resistance-heating vapor deposition method and the coating method.

Typical examples of the coating method include a spin coating method, a casting method, a dip coating method, and the like. A light-emitting layer is formed by dissolving or dispersing the light-emitting layer material above in a solvent and then coating the resulting solution or dispersion by the coating method. At this time, the material may be dissolved or dispersed together with a resin component. Examples of the resin components include polyvinyl chloride, polycarbonate, polystyrene, polymethyl methacrylate, polybutyl methacrylate, polyester, polysulfone, polyphenylene oxide, polybutadiene, poly(N-vinylcarbazole), hydrocarbon resins, ketone resins, phenoxy resins, polyamide, ethylcellulose, vinyl acetate resins, ABS resins, alkyd resins, epoxy resins, silicone resins, and the like.

The material for the hole injection layer and hole transporting layer is not particularly limited, if it has a function of receiving holes from the anode, a function of transporting the holes, or a function of blocking the electrons injected from the cathode. Typical examples thereof include, and are not limited to, carbazole derivatives, arylamine derivatives, styrylamine derivatives, phenylenediamine derivatives, amino-substituted chalcone derivatives, hydrazone derivatives, silazane derivatives, oxazole derivatives, imidazole derivatives, pyrazoline derivatives, pyrazolone derivatives, oxadiazole derivatives, triazole derivatives, polyarylalkane derivatives, stilbene derivatives, styrylanthracene derivatives, fluorenone derivatives, aromatic dimethylidene compounds, porphyrin derivatives, phthalocyanine derivatives, arylborane derivatives, arylsilane derivatives, conductive polymer or oligomer compounds such as poly(N-vinylcarbazole), aniline copolymers, polythiophenes, thiophene oligomers, polysilanes, and silane oligomers, the tetradentate ligands of the present invention, the platinum complexes of the present invention, and the like.

The thickness of the hole injection layer or the hole transporting layer is not particularly limited, and is selected in the range of preferably 1 nm to 5 μm, more preferably 5 nm to 1 μm, and still more preferably 10 to 500 nm. The hole injection layer or the hole transporting layer may have a single-layered structure of one or more of the materials described above or a multilayer structure having multiple layers same or different in composition. Examples of the method of forming the hole injection layer or the hole transporting layer include an electron beam method, a sputtering method, a resistance-heating vapor deposition method, a molecular lamination method, a coating method, an inkjet method, and a LB method, and the like; and preferred are the resistance-heating vapor deposition method and the coating method. In the coating method, a hole injection/transporting material may be dissolved or dispersed together with the resin component described above.

The material for the electron injection layer or the electron transporting layer is not particularly limited, if it has a function of receiving electrons from the cathode, a function of transporting the electrons, or a function of blocking the holes injected from the anode. When an electron injection/transporting material is used for blocking the holes injected from the anode, a material having higher ionization potential than that of the light-emitting layer is preferably selected.

Typical examples thereof include oxazole derivatives, oxadiazole derivatives, triazole derivatives, distyrylpyrazine derivatives, bipyridine derivatives, phenanthroline derivatives, carbodiimide derivatives, fluorenone derivatives, anthrone derivatives, diphenylquinone derivatives, thiopyranedioxide derivatives, anthraquinonedimethane derivatives, fluorenylidenemethane derivatives, aromatic tetracarboxylic acid anhydride derivatives, phthalocyanine derivatives, arylborane derivatives, arylsilane derivatives, various typical, transition or rare-earth metal complexes including metal complexes with an 8-quinolinol derivative, a benzoxazole derivative or a benzothiazole derivative as the ligand, polymer or oligomer compounds such as poly(N-vinylcarbazole), polythiophene, polyphenylene, and polyphenylene vinylene, the tetradentate ligands of the present invention, the platinum complexes of the present invention, and the like. Each of the polymer or oligomer compounds may have the tetradentate ligand of the present invention or the platinum complex of the present invention as its partial structure independently. The materials for the electron injection layer or the electron transporting layer are not limited to the materials above.

The thickness of the electron injection layer or the electron transporting layer is not particularly limited, and is selected in the range of preferably 1 nm to 5 μm, more preferably 5 nm to 1 μm, and still more preferably 10 nm to 500 nm. The electron injection layer or the electron transporting layer may have a single-layered structure of one or more of the materials described above or a multilayer structure having multiple layers same or different in composition. Examples of the method of forming the electron injection layer or the electron transporting layer include an electron beam method, a sputtering method, a resistance-heating vapor deposition method, a molecular lamination method, a coating method, an inkjet method, and a LB method, and the like; and preferable are the resistance-heating vapor deposition method and the coating method. In the coating method, solution or dispersion in which an electron injection/transporting material is dissolved or dispersed together with the resin component described above may be used.

The material for the protecting layer is not particularly limited, if it has a function of preventing molecules accelerating deterioration of the device such as water and oxygen from entering into the device. Typical examples thereof include metals such as indium, tin, lead, gold, silver, copper, aluminum, titanium and nickel; metal oxides such as magnesium oxide, silicon dioxide, dialuminum trioxide, germanium oxide, nickel oxide, calcium oxide, barium oxide, diiron trioxide, diytterbiumtrioxide and titanium oxide; metal fluorides such as lithium fluoride, magnesium fluoride, calcium fluoride, and aluminum fluoride; polymer compounds such as polyethylene, polypropylene, polymethyl methacrylate, polyimide, polyurea, polytetrafluoroethylene, polychloro-trifluoroethylene, and polydichlorodifluoroethylene; copolymer compounds such as a copolymer of chlorotrifluoroethylene and dichlorodifluoroethylene, copolymers obtained by copolymerization of a monomer mixture containing tetrafluoroethylene and at least one comonomer, and fluorine-containing copolymers having a cyclic structure on the main chain of copolymer; water-absorbing substances having a water absorption of 1% or more and moisture-proof substances having a water absorption of 0.1% or less, and the like.

The method of forming the protecting layer is also not particularly limited, and for example, methods such as a vacuum deposition method, a sputtering method, a reactive sputtering method, a MBE (molecular beam epitaxy) method, a cluster ion beam method, an ion plating) method, a plasma polymerization (high-frequency excitation ion plating) method, a plasma CVD method, a laser CVD method, a thermal CVD method, a gas source CVD method, and a coating method are applicable. From the viewpoint of color purity, the luminescence maximum wave (λmax) of the light-emitting device of the present invention is in the range of preferably 350 nm to 520 nm, more preferably 400 nm to 500 nm, and still more preferably 420 nm to 500 nm.

### EXAMPLES

Hereinafter, the present invention will be described in detail with reference to Examples, but it should be understood that the present invention is not limited thereby. In the Examples, the apparatuses used in determining physical properties are as follows: ¹H-NMR spectrum: DRX-500 (Bruker Japan Co., Ltd.)

Internal standard substance: tetramethylsilane or residual undeuterated solvent

EL peak: USB2000 (Ocean Optics, Inc.)

### Luminance: CS-100 (MINOLTA)

### Current and Voltage: source meter 2400 (Keithley Instruments Inc.)

### Example 1

### Preparation of 3-chloro-1-phenyl-1H-indazole

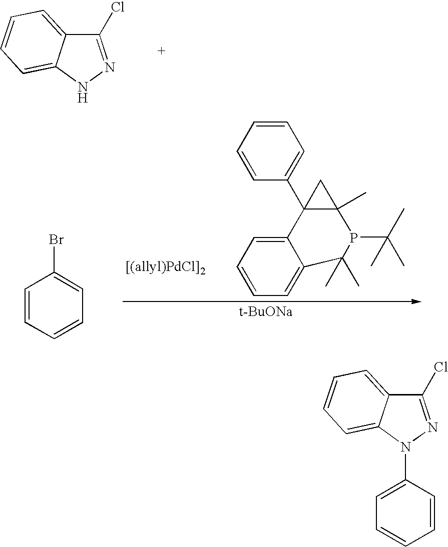

A mixture of 3-chloro-1H-indazole (1.50 g), bromobenzene (2.16 g), sodium t-butoxide (1.1 g), n-allylpalladium chloride (36 mg), di-t-butyl-(2,2-diphenyl-1-methylcyclopropyl)phosphine (139 mg) and xylene (40 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give 3-chloro-1-phenyl-1H-indazole as a viscous oil (1.62 g).

¹H-NMR (CDCl₃) δ: 7.26-7.40 (m, 2H), 7.46-7.57 (m, 3H), 7.69-7.78 (m, 4H).

### Example 2

### Preparation of 3-amino-1-phenylpyrazole

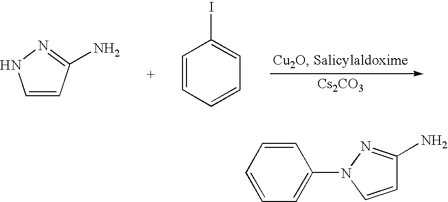

A mixture of 3-aminopyrazole (1.0 g), cesium carbonate (4.32 g), cuprous oxide (86.1 mg), salicylaldoxime (330 mg), iodobenzene (2.58 g) and N,N-dimethylformamide (8 mL) was stirred under nitrogen atmosphere at 95° C. for 16 hours. The reaction solution obtained was allowed to cool to room temperature. Water and toluene were added thereto and the extraction was carried out. The organic phases obtained were combined and concentrated. The residue obtained was purified by silica gel column chromatography to give 3-amino-1-phenylpyrazole as yellowish orange oil (1.26 g).

¹H-NMR (CDCl₃) δ: 3.81 (br, 2H), 5.85 (d, J=1.8 Hz, 1H), 7.18 (t, J=8.4 Hz, 1H), 7.36-7.60 (m, 4H), 7.69 (d, J=1.6 Hz, 1H).

### Example 3

### Preparation of N-(1-phenyl-1H-indazole-3-yl)aniline

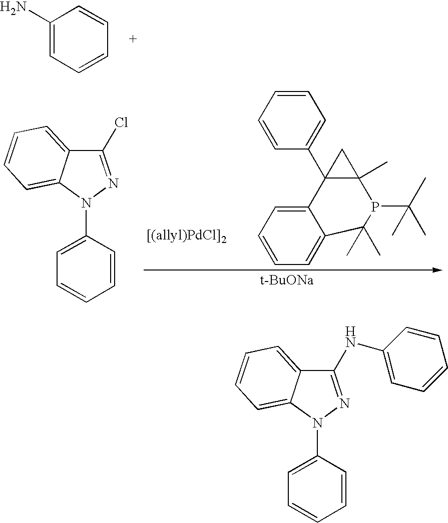

A mixture of 3-chloro-1-phenyl-1H-indazole (719 mg), aniline (139 mg), sodium t-butoxide (317 mg), π-allylpalladium chloride (11 mg), di-t-butyl-(2,2-diphenyl-1-methylcyclopropyl) phosphine (42 mg) and xylene (20 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give N-(1-phenyl-1H-indazole-3-yl)aniline as a viscous oil (280 mg).

¹H-NMR (CDCl₃) δ: 6.36 (br, 1H), 6.97 (t, J=4.8 Hz, 1H), 7.17 (t, J=4.8 Hz, 1H), 7.26-7.57 (m, 8H), 7.64 (d, J=5.4 Hz, 1H), 7.75-7.79 (m, 3H).

### Example 4

### Preparation of N-(1-phenyl-1H-indazole-3-yl)-N-(6-phenylpyridine-2-yl)aniline

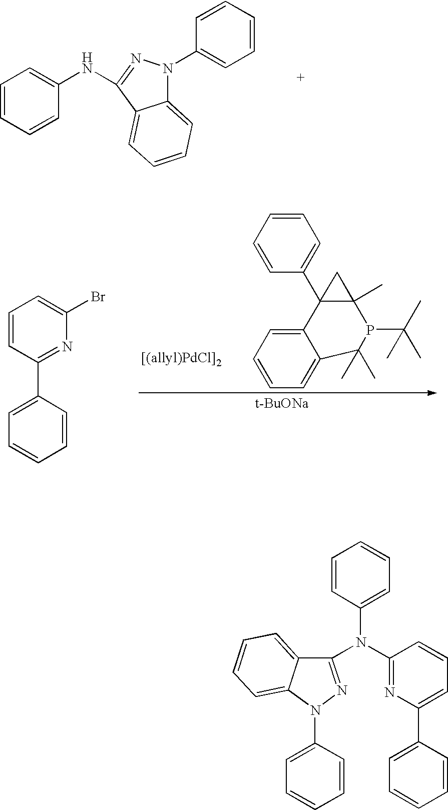

A mixture of N-(1-phenyl-1H-indazole-3-yl)aniline (400 mg), 2-bromo-6-phenylpyridine (430 mg), sodium t-butoxide (185 mg), π-allylpalladium chloride (13 mg), di-t-butyl-(2,2-diphenyl-1-methylcyclopropyl)phosphine (49 mg) and xylene (20 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give N-(1-phenyl-1H-indazole-3-yl)-N-(6-phenylpyridine-2-yl)aniline as a viscous oil (412 mg).

¹H-NMR (CDCl₃) δ: 6.86 (d, J=5.4 Hz, 1H), 7.06 (t, J=5.0 Hz, 1H), 7.16-7.60 (m, 15H), 7.71-7.81 (m, 5H).

### Example 5

### Preparation of Platinum Complex

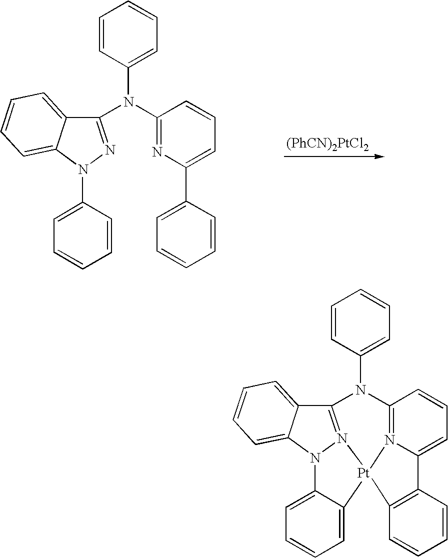

A mixture of N-(1-phenyl-1H-indazole-3-yl)-N-(6-phenylpyridine-2-yl)aniline (412 mg), dichlorobis(benzonitrile)platinum (444 mg) and xylene (14 mL) was stirred in reflux condition under nitrogen atmosphere for 2 hours. The solvent in the reaction solution was distilled off, and the residue obtained was purified by silica gel column chromatography and crystallized (ethanol/CH₂Cl₂), to give an objective platinum complex as yellow powder (335 mg).

¹H-NMR (DMSO-d₆) δ: 5.60 (d, J=5.8, 1H), 6.97 (d, J=5.6 Hz, 1H), 7.06 (t, J=4.8 Hz, 1H), 7.20-7.47 (m, 4H), 7.71 (t, J=5.2 Hz, 1H), 7.85-7.98, m, 5H), 8.11-8.21 (m, 4H), 8.34 (d, J=5.0 Hz, 1H), 8.45 (d, J=4.0 Hz, 1H), 8.53 (d, J=6.0 Hz, 1H).

### Example 6

Preparation of N-(1-phenylpyrazole-3-yl)-4-t-butylaniline

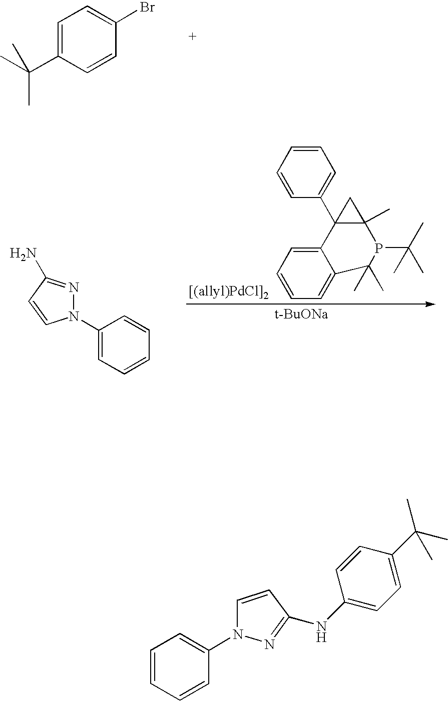

A mixture of 3-amino-1-phenylpyrazole (700 mg), 4-bromo-t-butylbenzene (1.02 g), sodium t-butoxide (465 mg), n-allylpalladium chloride (32.2 mg), di-t-butyl-(2,2-diphenyl-1-methylcyclopropyl) phosphine (124 mg) and xylene (20 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give N-(1-phenylpyrazole-3-yl)-4-t-butylaniline as a viscous oil (780 mg).

¹H-NMR (CDCl₃) δ: 1.27 (s, 9H), 6.11 (br, 1H), 6.17 (d, J=1.6 Hz, 1H), 7.16-7.66 (m, 9H), 7.80 (d, J=1.6 Hz, 1H).

### Example 7

### Preparation of N-(1-phenylpyrazole-3-yl)-N-(6-phenylpyridine-2-yl)-4-t-butylaniline

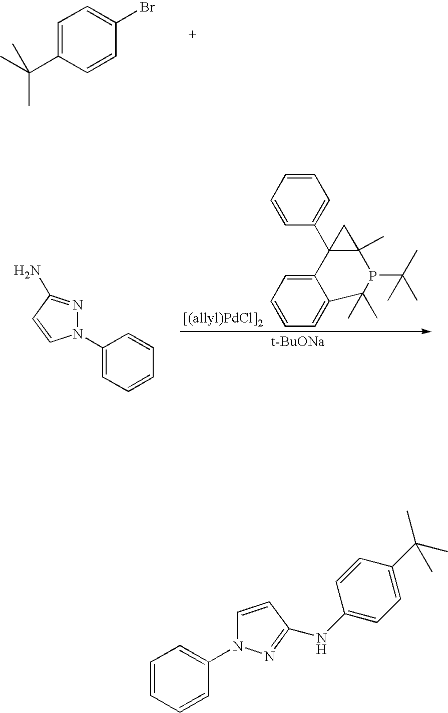

A mixture of N-(1-phenylpyrazole-3-yl)-4-t-butylaniline (351 mg), 2-bromo-6-phenylpyridine (234 mg), sodium t-butoxide (115 mg), π-allylpalladium chloride (9.10 mg), di-t-butyl-(2,2-diphenyl-1-methylcyclopropyl)phosphine (35.3 mg) and xylene (15 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give N-(1-phenylpyrazole-3-yl)-N-(6-phenylpyridine-2-yl)-4-t-butylaniline as a viscous oil (282 mg).

¹H-NMR (CDCl₃) δ: 1.35 (s, 9H), 6.48 (d, J=1.8 Hz, 1H), 6.87 (d, J=5.2 Hz, 1H), 7.26-7.44 (m, 10H), 7.51 (t, J=5.6 Hz, 1H), 7.62-7.66 (m, 3H), 7.89-7.96 (m, 3H).

### Example 8

### Preparation of Platinum Complex

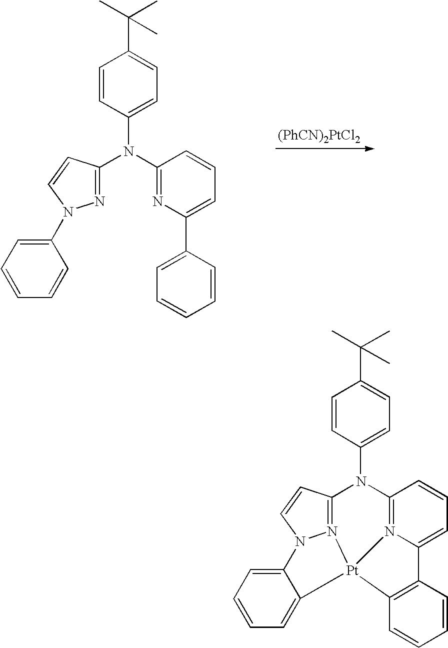

A mixture of N-(1-phenylpyrazole-3-yl)-N-(6-phenylpyridine-2-yl)-4-t-butylaniline (140 mg), dichlorobis(benzonitrile)platinum (150 mg), acetic acid (5 ml) and xylene (5 mL) was stirred in reflux condition under nitrogen atmosphere for 2 hours. The solvent in the reaction solution was distilled off, and the residue obtained was purified by silica gel column chromatography and crystallized (ethanol/CH₂Cl₂), to give an objective platinum complex as yellowish orange powder (48 mg).

¹H-NMR (DMSO-d₆) δ: 1.42 (s, 9H), 5.69 (d, J=2.2 Hz, 1H), 6.74 (d, J=5.8 Hz, 1H), 7.18 (t, J=5.2 Hz, 1H), 7.24-7.27 (m, 2H), 7.34 (t, J=4.8 Hz, 1H), 7.63 (d, J=5.8 Hz, 2H), 7.78-7.88 (m, 3H), 8.03 (t, J=5.0 Hz, 1H), 8.12 (t, J=5.6 Hz, 1H), 8.22 (d, J=5.0 Hz, 1H), 8.27-8.30 (m, 1H), 8.85 (d, J=1.8 Hz, 1H).

### Example 9

### Preparation of N-(1-phenylpyrazole-3-yl)-N-(1-phenyl-1H-indazole-3-yl)-4-t-butylaniline

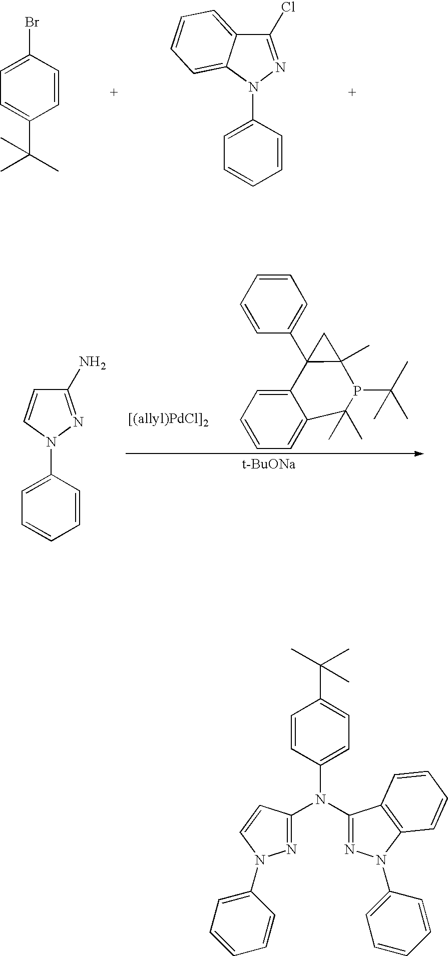

A mixture of 3-amino-1-phenylpyrazole (435 mg), 3-chloro-1-phenyl-1H-indazole (750 mg), sodium t-butoxide (630 mg), π-allylpalladium chloride (45.0 mg), di-t-butyl-(2,2-diphenyl-1-methylcyclopropyl)phosphine (173 mg) and xylene (25 mL) was stirred under a nitrogen atmosphere at 95° C. for 3 hours. Then, 4-bromo-t-butylbenzene (582 mg) was added to the reaction mixture, further stirring at 95° C. for overnight. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give N-(1-phenylpyrazole-3-yl)-N-(1-phenyl-1H-indazole-3-yl)-4-t-butylaniline as a viscous oil (859 mg).

¹H-NMR (CDCl₃) δ: 1.35 (s, 9H), 6.33 (d, J=1.6 Hz, 1H), 6.97-7.09 (m, 2H), 7.25-7.40 (m, 9H), 7.49 (t, J=5.4 Hz, 2H), 7.57-7.61 (m, 2H), 7.71-7.76 (m, 3H), 7.86 (d, J=1.6 Hz, 1H).

### Example 10

### Preparation of Platinum Complex

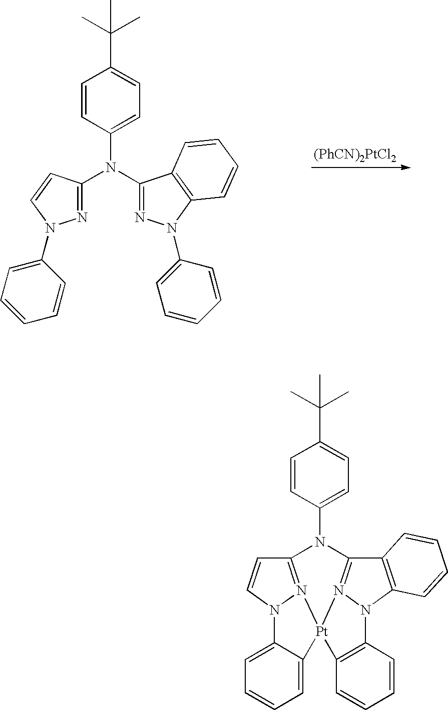

A mixture of N-(1-phenylpyrazole-3-yl)-N-(1-phenyl-1H-indazole-3-yl)-4-t-butylaniline (430 mg), dichlorobis(benzonitrile)platinum (432 mg), acetic acid (4 ml) and xylene (4 mL) was stirred in reflux condition under nitrogen atmosphere for 2 hours. The solvent in the reaction solution was distilled off, and the residue obtained was purified by silica gel column chromatography and crystallized (ethanol/CH₂Cl₂), to give a objective platinum complex as yellow powder (21 mg).

¹H-NMR (DMSO-d₆) δ: 1.44 (s, 9H), 6.03 (d, J=5.6 Hz, 1H), 6.22 (d, J=2.0 Hz, 1H), 7.10 (t, J=5.2 Hz, 1H), 7.22-7.38 (m, 4H), 7.73 (t, J=5.2 Hz, 1H), 7.85 (s, 4H), 7.97-8.00 (m, 1H), 8.21-8.36 (m, 3H), 8.56 (d, J=6.0 Hz, 1H), 9.03 (d, J=1.8 Hz, 1H).

### Example 11

### Preparation of 3-(1-phenylpyrazole-3-yl)amino-1-phenyl-5-methylpyrazole

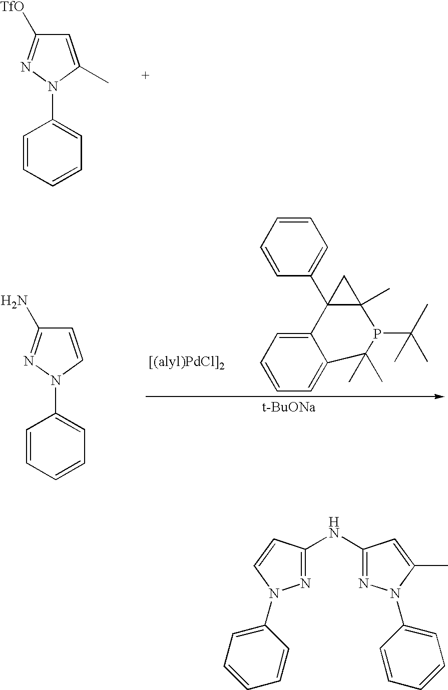

A mixture of 3-trifluoromethanesulfonyloxy-5-methyl-1-phenylpyrazole (500 mg), 3-amino-1-phenylpyrazole (obtained by above Example 2)(261 mg), sodium t-butoxide (164 mg), π-allylpalladium chloride (23.8 mg), di-t-butyl-(2,2-diphenyl-1-methylcyclopropyl)-phosphine (91.8 mg) and xylene (20 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give 3-(1-phenylpyrazole-3-yl) amino-1-phenyl-5-methylpyrazole as a viscous oil (200 mg).

¹H-NMR (CDCl₃) δ: 2.35 (s, 3H), 6.25 (s, 1H), 6.33 (d, J=1.8 Hz, 1H), 6.58 (br, 1H), 7.20-7.50 (m, 8H), 7.62-7.67 (m, 2H), 7.79 (d, J=1.8 Hz, 1H).

### Example 12

### Preparation of N-(1-phenylpyrazole-3-yl)-N-(1-phenyl-5-methylpyrazole-3-yl)-4-(di(p-tolyl)amino)aniline

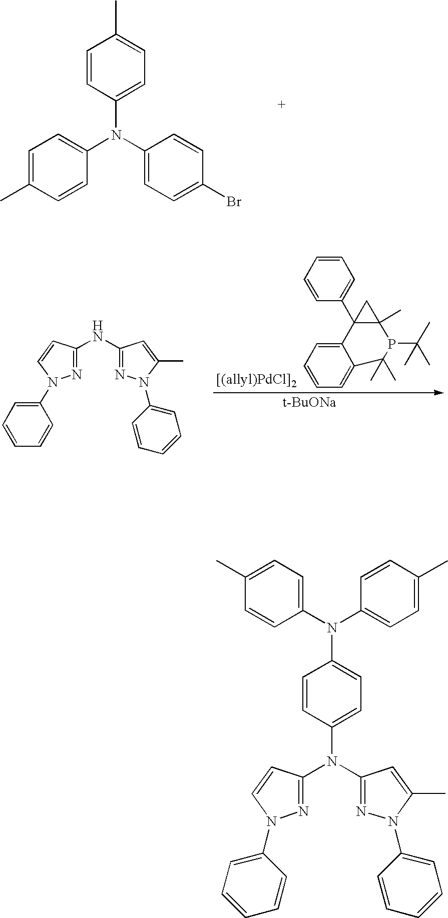

A mixture of 3-(1-phenylpyrazole-3-yl)amino-1-phenyl-5-methylpyrazole (obtained by above Example 11)(350 mg), N,N-di(p-tolyl)-4-bromoaniline (390 mg), sodium t-butoxide (112 mg), π-allylpalladium chloride (8.1 mg), di-t-butyl-(2,2-diphenyl-1-methylcyclopropyl)phosphine (31.3 mg) and xylene (10 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give N-(1-phenylpyrazole-3-yl)-N-(1-phenyl-5-methylpyrazole-3-yl)-4-(di(p-tolyl)amino)aniline as a viscous oil (349 mg).

¹H-NMR (CDCl₃) δ: 2.30-2.38 (m, 9H), 6.07 (s, 1H), 6.31 (d, J=1.8 Hz, 1H), 6.94-7.45 (m, 20H), 7.60-7.63 (m, 2H), 7.81 (d, J=1.6 Hz, 1H).

### Example 13

### Preparation of Platinum Complex

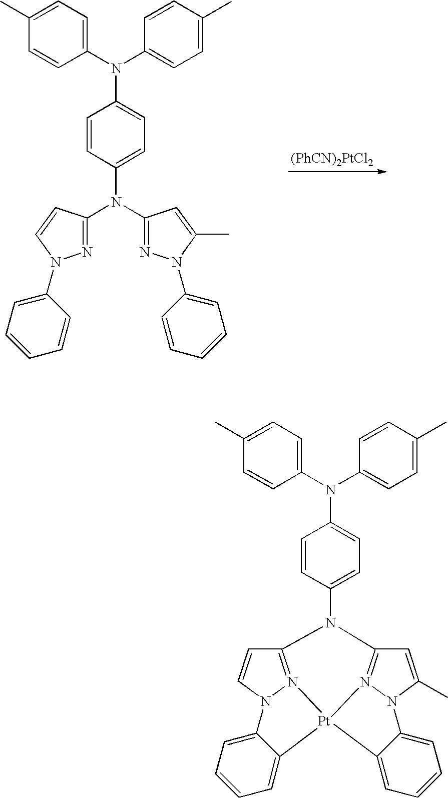

A mixture of N-(1-phenylpyrazole-3-yl)-N-(1-phenyl-5-methylpyrazole-3-yl)-4-(di (p-tolyl)amino) aniline (obtained by above Example 12) (308 mg), dichlorobis(benzonitrile)platinum (248 mg), acetic acid (4 ml) and xylene (4 mL) was stirred in reflux condition under nitrogen atmosphere for 2 hours. After cooling the reaction mixture, the mixture was concentrated. An aqueous sodium carbonate solution was added to the concentrate and the mixture was extracted with methylenedichloride. The solvent of the organic layer was distilled off, and the residue obtained was purified by silica gel column chromatography and crystallized (heptane/isopropanol), to give an objective platinum complex as white powder (53 mg).

¹H-NMR (DMSO-d₆) δ: 2.31 (s, 6H), 2.83 (s, 3H), 6.08 (s, 1H), 6.21 (d, J=1.6 Hz, 1H), 7.05-7.58 (m, 16H), 7.72-7.90 (m, 2H), 8.17-8.28 (m, 2H), 8.92 (d, J=1.8 Hz, 1H).

### Example 14

### Preparation of N-(1-phenylpyrazole-3-yl)-N-(1-phenyl-5-methylpyrazole-3-yl)-3,5-di-t-butylaniline

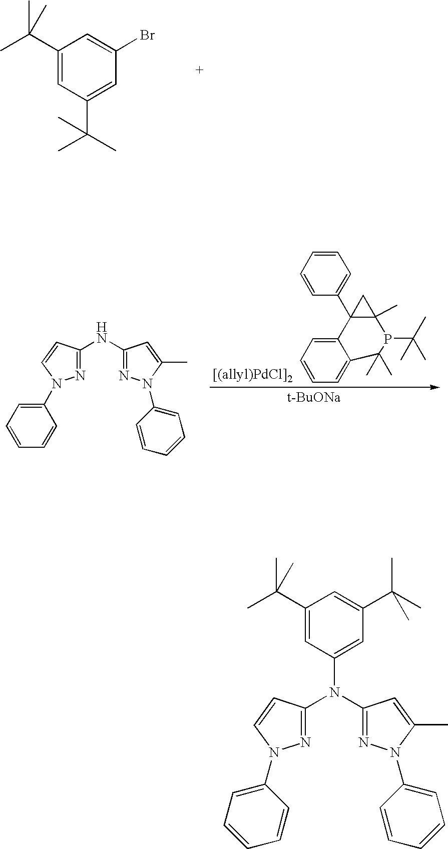

A mixture of 3-(1-phenylpyrazole-3-yl)amino-1-phenyl-5-methylpyrazole (obtained by above Example 11)(200 mg), 3,5-di-t-butylbromobenzene (171 mg), sodium t-butoxide (67.0 mg), π-allylpalladium chloride (8.1 mg), di-t-butyl-(2,2-diphenyl-1-methylcyclopropyl)phosphine (31.3 mg) and xylene (10 mL) was stirred under a nitrogen atmosphere at 95° C. for 3 hours. The reaction solution was allowed to cool to room temperature. Then aqueous ammonium chloride-saturated solution was added thereto and the mixture was extracted with toluene. The organic phases obtained were combined and concentrated, and the residue obtained was purified by silica gel column chromatography to give N-(1-phenylpyrazole-3-yl)-N-(1-phenyl-5-methylpyrazole-3-yl)-3,5-di-t-butylaniline as a viscous oil (206 mg).

¹H-NMR (CDCl₃) δ: 1.30 (s, 18H), 2.36 (s, 3H), 6.09 (s, 1H), 6.29 (d, J=1.8 Hz, 1H), 7.15-7.49 (m, 11H), 7.61-7.64 (m, 2H), 7.80 (d, J=1.8 Hz, 1H).

### Example 15

### Preparation of Organic EL Device

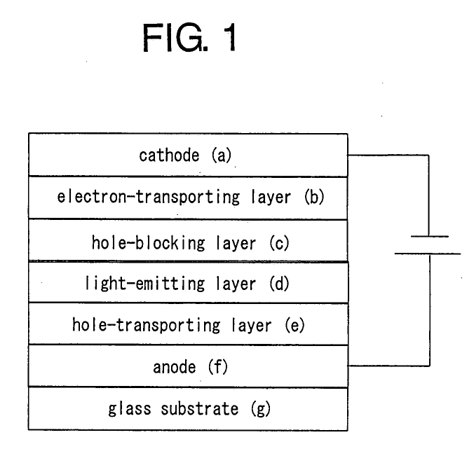

An organic EL device having the layer structure shown in FIG. 1 was prepared by forming an anode (f), a hole transporting layer (e), a light-emitting layer (d) comprising a host material and a dope material, a hole blocking layer (c), an electron transporting layer (b) and a cathode (a) on a glass plate (g), sequentially in the order from the glass plate (g) side. In the organic EL device, each of the anode (f) and the cathode (a) is connected to a lead wire, and voltage can be applied between the anode (f) and cathode (a). Specific materials and preparative methods for each layer will be described below.

The anode (f) is an ITO film and bonded onto the glass plate (g). The hole transporting layer (e) was formed on the anode (f) to a thickness of 40 nm by vacuum deposition of 4,4′-bis[N-(1-naphthyl)-N-phenylamino]biphenyl (α-NPD) represented by the following Formula:

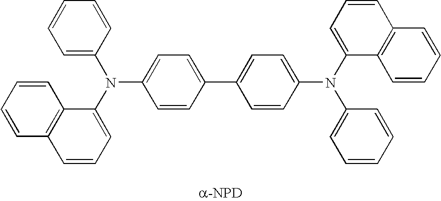

The light-emitting layer (d) containing a host material and a doped phosphorescent luminous material was formed on the hole transporting layer (e) to a thickness of 35 nm by vacuum co-deposition of 4,4′-bis(9H-carbazole-9-yl) biphenyl (CBP) and the platinum complex obtained in Example 5 (platinum complex-doping amount: 1 wt-%) represented by the following Formulae:

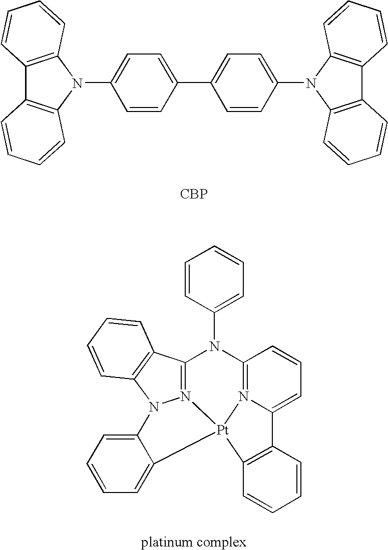

The hole blocking layer (c) was formed on the light-emitting layer (d) to a thickness of 10 nm by vacuum deposition of 2,9-dimethyl-4,7-diphenyl-1,10-phenanthroline (BCP) represented by the following Formula:

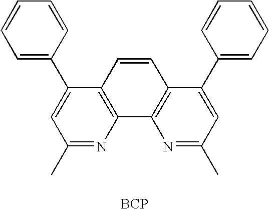

The electron transporting layer (b) was formed on the hole blocking layer (c) to a thickness of 35 nm by vapor deposition of tris (8-quinolinolato-O,N] aluminum (Alq₃) represented by the following Formula:

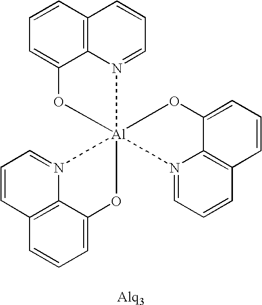

The cathode (a) was formed as a laminate film by vacuum deposition of lithium fluoride to a thickness of 0.5 nm and metal aluminum to a thickness of 100 nm, sequentially from the electron transporting layer (b) side.

Application of a plus voltage to the anode (f) and a minus voltage to the cathode (a) of the organic EL device thus prepared resulted in stabilized blue green emission derived from the platinum complex obtained in Example 5. The external quantum efficiency of the device at a brightness of 100 cd/m² was 1.6 (%).

### Example 16

An organic EL device having an device configuration similar to that in Example 15 was prepared in a similar manner to Example 15, except that there were used in the light-emitting layer (d) 4,4′-bis(9H-carbazole-9-yl)-2,2′-dimethylbiphenyl (CDBP) and the platinum complex obtained in Example 8 represented by the following Formulae:

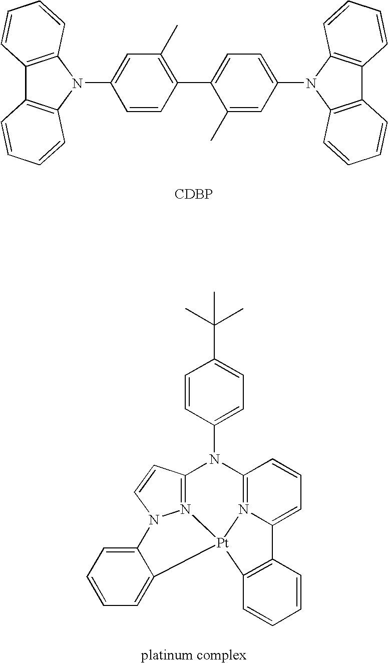

Physical property of the device was determined in a similar manner to Example 15 at a brightness of 100 cd/m², the external quantum efficiency of the device was 2.5 (%), the device gave a blue green emission derived from the platinum complex obtained in Example 8.

### Example 17

An organic EL device having an device configuration similar to that in Example 16 was prepared in a similar manner to Example 16, except that there was used in the light-emitting layer (d) as a phosphorous luminous material the platinum complex obtained in Example 10 represented by the following Formula:

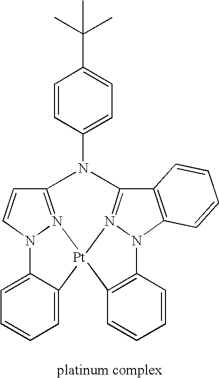

Physical property of the device was determined in a similar manner to Example 16 at a brightness 100 cd/m², the external quantum efficiency of the device was 0.5 (%), the device gave a blue green emission derived from the platinum complex obtained in Example 10.

Results obtained in Examples 15 to 17 are summarized in the following Tables 1.

TABLE 1
Characteristics of the EL device prepared

| | Emission | External quantum | Power efficiency |
|---|---|---|---|
| | peak | efficiency at 100 cd/m² | at 100 cd/m² |
| Ex. No. | (nm) | (%) | (lm/W) |
| 15 | 497 | 1.6 | 1.1 |
| 16 | 498 | 2.5 | 2.1 |
| 17 | 485 | 0.5 | 0.3 |

What is claimed is:

1. A platinum complex represented by General Formula (1): 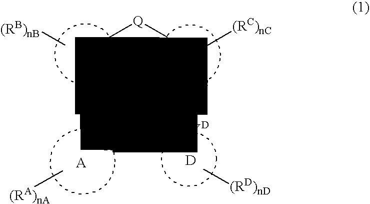
wherein ring A represents a six-membered aromatic ring or a six-membered aromatic heterocyclic ring; ring B represents a five-membered nitrogen-containing aromatic heterocyclic ring; rings C and D each independently represent an aromatic ring or an aromatic heterocyclic ring, while either of the ring C or D represents five-membered ring, the other represents a five- or six-membered ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C} or R^{D} to form a fused ring independently; two of X^{A}, X^{B}, X^{C}, and X^{D} represent a nitrogen atom that may be bound with the platinum atom by a coordinate bond, while the other two each independently represent a carbon atom or nitrogen atom that may be bound with the platinum atom by a covalent bond; Q represents a bivalent atom or atomic group bridging the rings B and C; the ring B and Q, and the ring C and Q each independently may be bound each other via a substituent R^{B} or R^{C} to form a fused ring; Y^{C} and Y^{D} each independently represent a carbon atom or a nitrogen atom; nA is an integer of 0 to 4, nB is an integer of 0 to 2, nC is an integer of 0 to 2 when the ring C is five-membered ring or an integer of 0 to 3 when the ring C is six-membered ring, and nD is an integer of to 3 when the ring D is five-membered ring or an integer of 0 to 4 when the ring D is six-membered ring; and when nA, nB, nC or nD is 2 or more, the groups R^{A}, the groups R^{B}, the groups R^{C}, and the groups R^{D} each independently may be bound each other to form a fused ring.

2. A light-emitting device containing one or more of the compound represented by General Formula (1) described in claim 1.

3. The light-emitting device according to claim 2, wherein the light-emitting device is one having a light-emitting layer or plural organic compound layers including a light-emitting layer between a pair of electrodes and contains one or more of compounds represented by General Formula (1) in one or more of the layers.

4. The light-emitting device according to claim 3, wherein the light-emitting device is an organic electroluminescent device (organic EL device).

5. The light-emitting device according to claim 4, wherein the compound represented by General Formula (1) and contained in one or more of the layers is able to act as a doping material (guest material) in the light-emitting material of the organic electroluminescent device.

6. A compound represented by General Formula (2): 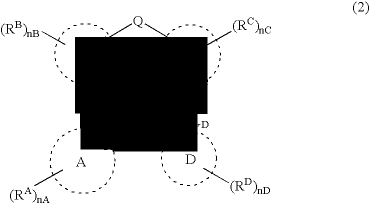
wherein ring A represents a six-membered aromatic ring or a six-membered aromatic heterocyclic ring; ring B represents a five-membered nitrogen-containing aromatic heterocyclic ring; rings C and D each independently represent an aromatic ring or an aromatic heterocyclic ring, while either of the ring C or D represents five-membered ring, the other represents a five- or six-membered ring; R^{A}, R^{B}, R^{C}, and R^{D} respectively represent substituents on the rings A, B, C, and D; the rings A and B, the rings B and C, and the rings C and D each may be bound each other via the substituent R^{A}, R^{B}, R^{C}, or R^{D} to form a fused ring independently; two of X^{A′}, X^{B′}, X^{C′}, and X^{D′} represent a nitrogen atom having a lone electron pair, while the other two represent CH or NH; Q represents a bivalent atom or atomic group bridging the rings B and C; and the ring B and Q, and the ring C and Q each independently may be bound each other via a substituent R^{B} or R^{C} to form a fused ring; Y^{C} and Y^{D} represent a carbon atom or a nitrogen atom; nA is an integer of 0 to 4, nB is an integer of 0 to 2, nC is an integer of 0 to 2 when the ring C is five-membered ring or an integer of 0 to 3 when the ring C is six-membered ring, and nD is an integer of 0 to 3 when the ring D is five-membered ring or an integer of 0 to 4 when the ring D is six-membered ring; and when nA, nB, nC or nD is 2 or more, the groups R^{A}, the groups R^{B}, the groups R^{C}, and the groups R^{D} each independently may be bound each other to form a fused ring.
